(19) United States
(12) Patent Application Publication
(10) Pub. No.: US 2026/0120827 A1
(43) Pub. Date: Apr. 30, 2026

(54) SYSTEMS AND METHODS FOR GENERATING CLINICAL HANDOFF SUMMARIES

(71) Applicant: Oracle International Corporation, Redwood Shores, CA (US)

(72) Inventors: Yuwen Zhang, Kansas City, KS (US); Zachary McBride Lazri, Baltimore, MD (US); Andrew Roberts, Overland Park, KS (US); Joseph James O'Malley, Mission, KS (US); Jacob Geers, Kansas City, KS (US)

(73) Assignee: Oracle International Corporation, Redwood Shores, CA (US)

(21) Appl. No.: 19/368,835

(22) Filed: Oct. 24, 2025

Related U.S. Application Data
(60) Provisional application No. 63/712,370, filed on Oct. 25, 2024.

(51) Int. Cl. G16H 10/60; G06F 16/9038

(52) U.S. Cl. CPC G16H 10/60; G06F 16/9038

(57) ABSTRACT

A computer-implemented method includes receiving a query to provide a summary of patient-specific information regarding a condition for a particular patient. The method includes determining a category for the query, retrieving data relevant to the query from an electronic health record (EHR) database, including at least structured and unstructured content, and processing and filtering the data as retrieved based on the category. The method further includes generating, by a generative machine learning model, a narrative summary including a first portion of filtered data and some of the unstructured content, generating a structured summary including a second portion of filtered data, including some of the structured content, and formatting the narrative summary and the structured summary into an output. Determining the category for the query includes selecting the category from a plurality of categories, and processing performed for a first category differs from processing performed for a second category.

### CROSS-REFERENCES TO RELATED APPLICATIONS

The present application is a non-provisional application of and claims the benefit and priority to U.S. Provisional Application No. 63/712,370, filed Oct. 25, 2024, the entire contents of which is incorporated herein by reference for all purposes.

### BACKGROUND

Traditional healthcare systems involving computer-based assistants often rely on retrieving information from multiple data sources such as electronic health records and other data sources. Often these data sources code this data using custom coding schemes. As a result, retrieving and formatting retrieved information for standardized assistant user interfaces can be challenging. Traditional Electronic Health Record (EHR) systems can also have complex interfaces that may be difficult to navigate and cumbersome to operate. When the process for retrieving or recording necessary patient information is inefficient, it can disrupt the natural flow of the patient interaction.

Solutions addressing these changes and others would be desirable.

### SUMMARY

Techniques disclosed herein pertain to generative artificial intelligence (AI) systems, and, more specifically, to summary generation techniques using agentic AI systems.

In embodiments, a computer-implemented method includes receiving, by a computer, a query to provide a summary of patient-specific information regarding a condition for a particular patient. The method includes determining, by the computer, a category for the query, and retrieving, by the computer, data relevant to the query from an electronic health record (EHR) database, where the data as retrieved includes at least structured and unstructured content. The method further includes processing and filtering, by the computer, the data as retrieved based on the category. The method further includes generating, by a generative machine learning model on the computer, a narrative summary including a first portion of the data as filtered, including at least a portion of the unstructured content, and generating, by the computer, a structured summary including a second portion of the data as filtered, including at least a portion of the structured content. The method further includes formatting, by the computer, the narrative summary and the structured summary into an output. In embodiments, determining the category for the query includes selecting the category from a plurality of categories, including at least one of New Admit, New to Me, and Rounded on Before, and processing performed for a first selected category differs from processing performed for a second selected category.

In certain embodiments, the method includes, for the first category, providing a first set of processing modules, and, for the second category, providing a second set of processing modules. In embodiments, filtering the data includes considering the data as processed according to a semantic knowledge graph selected based on the category. In certain embodiments, the method includes processing the data according to the semantic knowledge graph by applying enrichment that prioritizes selected data in a predefined hierarchy model based on the category and at least one of a reason for visit and a chief complaint for the particular patient. In embodiments, the predefined hierarchy model includes prioritizing data related to changes in the condition for the particular patient during a predetermined time window.

In embodiments, the method further includes transforming the data into an intermediate representation normalized to clinical terminologies, and filtering the intermediate representation to meet a token budget for the generative machine learning model on the computer. In certain embodiments, the method includes caching the intermediate representation as keyed to at least a selected one of the particular patient, the category, and a time window, and reusing the cache in responding to updated queries related to the particular patient and the category. In embodiments, the method includes processing the unstructured content through at least one of optical character recognition, image recognition, and chunking processes. In certain embodiments, the method further includes determining a role of an originator of the query, the role determining a level of permissions assigned to the originator, and processing is modified according to the role as determined.

In embodiments, a system includes a computer comprising one or more processors and one or more computer-readable media storing instructions which, when executed by the one or more processors, cause the computer to at least receive a query to provide a summary of patient-specific information regarding a condition for a particular patient. The system includes determining a category for the query, where determining includes selecting the category from a plurality of categories including at least one of New Admit, New to Me, and Rounded on Before. The system further includes retrieving data relevant to the query from an electronic health record (EHR) database, where the data as retrieved includes at least structured and unstructured content. The system further includes processing and filtering the data as retrieved based on the category. The system further includes generating, by a generative machine learning model on the computer, a narrative summary including a first portion of the data as filtered including at least a portion of the unstructured content, generating a structured summary including a second portion of the data as filtered including at least a portion of the structured content, and formatting the narrative summary and the structured summary into an output. The system further includes that processing performed for a first selected category differs from processing performed for a second selected category.

In certain embodiments, the system includes, for the first category, providing a first set of processing modules, and for the second category, providing a second set of processing modules. In embodiments, filtering includes considering the data as processed according to a semantic knowledge graph and a hierarchy model selected based on the category by applying enrichment that prioritizes selected data based on the category and at least one of a reason for visit and a chief complaint for the particular patient. In certain embodiments, the hierarchy model includes prioritization of data related to changes in the condition for the particular patient during a predetermined time window. In embodiments, the system further includes transforming the data into an intermediate representation normalized to clinical terminologies, and filtering the intermediate representation to meet a token budget for the generative machine learning model. In certain embodiments, the system includes caching the intermediate representation as keyed to at least a selected one of the particular patient, the category, and a time window, and reusing the cache in responding to updated queries related to the particular patient and the category. In embodiments, the system includes, for the unstructured content, processing the unstructured content through at least one of optical character recognition, image recognition, and chunking processes.

In embodiments, one or more non-transitory computer-readable media store instructions which, when executed by one or more processors on a computer, cause the computer to at least receive a query to provide a summary of patient-specific information regarding a condition for a particular patient. The instructions include determining a category for the query, where determining includes selecting the category from a plurality of categories including at least one of New Admit, New to Me, and Rounded on Before. The instructions further include retrieving data relevant to the query from an electronic health record (EHR) database, where the data as retrieved includes at least structured and unstructured content. The instructions further include processing and filtering the data as retrieved based on the category. The instructions further include generating, by a generative machine learning model on the computer, a narrative summary including a first portion of the data as filtered including at least a portion of the unstructured content, generating a structured summary including a second portion of the data as filtered including at least a portion of the structured content, and formatting the narrative summary and the structured summary into an output. The instructions further include that processing performed for a first selected category differs from processing performed for a second selected category.

In certain embodiments, the instructions include, for the first category, providing a first set of processing modules, and for the second category, providing a second set of processing modules. In embodiments, filtering includes considering the data as processed according to a semantic knowledge graph and a hierarchy model selected based on the category by applying enrichment that prioritizes selected data based on the category and at least one of a reason for visit and a chief complaint for the particular patient. In certain embodiments, the instructions include transforming the data into an intermediate representation normalized to clinical terminologies, and filtering the intermediate representation to meet a token budget for the generative machine learning model.

### BRIEF DESCRIPTION OF THE DRAWINGS

The present disclosure is described in conjunction with the appended figures, where like components are indicated with like reference numbers.

FIG. 1 is a block diagram illustrating an example computing environment incorporating agent-driven services, according to certain environments.

FIG. 2 is a block diagram illustrating an example of the function of portions of the cloud service provider platform of FIG. 1 in producing a clinical handoff summary, in accordance with embodiments.

FIG. 3 is an example clinical handoff summary layout, in accordance with embodiments.

FIG. 4 is a block diagram illustrating an alternative example of the function of portions of the cloud service provider platform of FIG. 1 in producing a clinical handoff summary, in accordance with embodiments.

FIG. 5 is a simplified diagram illustrating an example of a timeline in producing a series of clinical handoff summaries, in accordance with embodiments.

FIG. 6 is a flowchart illustrating an example process for generating a clinical handoff summary in accordance with a user-provided query, in accordance with embodiments.

FIG. 7 is a flowchart illustrating an alternative process for generating a clinical handoff summary in accordance with a user-provided query, in accordance with embodiments.

FIG. 8 shows a simplified diagram depicting a computing environment 800 incorporating an agent-driven digital assistant system configured to generate knowledge-grounded response data according to certain embodiments.

FIG. 9 is a block diagram illustrating one pattern for implementing a cloud infrastructure as a service system, according to at least one embodiment.

FIG. 10 is a block diagram illustrating another pattern for implementing a cloud infrastructure as a service system, according to at least one embodiment.

FIG. 11 is a block diagram illustrating another pattern for implementing a cloud infrastructure as a service system, according to at least one embodiment.

FIG. 12 is a block diagram illustrating another pattern for implementing a cloud infrastructure as a service system, according to at least one embodiment.

FIG. 13 is a block diagram illustrating an example computer system, according to at least one embodiment.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

Healthcare providers often find it useful to locate and review a variety of information regarding a patient prior to an encounter with the patient. Often, healthcare providers locate and review this information when assuming responsibility for a patient from another healthcare provider. For example, for patients admitted in a hospital setting, the care team typically transfers information about patients at shift overlaps and during handoffs between different members of the care team. Such clinical handoffs may occur several times a day during a patient's hospital stay. As a result, efficient retrieval of patient-specific information between healthcare providers and accurate knowledge transfer between healthcare providers is important to providing high quality patient care. However, locating, reviewing, and assembling the appropriate information for these handoffs can be difficult even with the proliferation of electronically accessible EHR systems. In many cases, to provide the information, healthcare providers often obtain information from disparate sources including EHR systems and spend time reviewing, organizing, and assembling the information such that it will be useful.

Electronic and computerized tools have been developed and utilized by healthcare providers to perform these tasks, but these tools often lack the computational resources to perform these tasks with low latency and high accuracy. One challenge often encountered by these tools is the different coding schemes employed by different information storage sources. For example, many EHR systems use proprietary coding systems for storing patient information. Additionally, these tools often lack the capabilities to generate customized information based on patient status and/or healthcare provider status (e.g., a patient new to healthcare provider, a newly admitted patient, a new healthcare provider for the patient).

In many cases, intelligent tools such as agentic digital assistants have employed to perform these tasks. These agentic digital assistants often utilize one or more generative machine learning models such as large language models (LLMs) to retrieve information related to an inquiry, process the information, and generate a response to the inquiry from the processed information. An example of such an approach is discussed in U.S. patent application Ser. No. 18/624,472, filed Apr. 2, 2024, which is incorporated herein by reference as if fully set forth herein.

While these agentic digital assistants have been useful in improving information retrieval and synthesis, utilizing these assistants in clinical settings presents challenges. For example, EHRs often encompass extensive and fragmented information, including personal information, patient histories, test results, physician notes, and medication records stored using different coding schemes, although processing this vast context efficiently poses a significant challenge for a variety of reasons such as information overload, model limitations, temporal context, and patient-specific context. In another example, EHR data is rarely presented in a unified format with both structured fields (e.g., lab results, medication lists) and unstructured text (e.g., physician notes, patient complaints), and processing these different formats poses data fusion and aggregation challenges, semantic alignment challenges, and inconsistencies across healthcare providers. In yet another example, LLMs and other generative machine learning models are often pre-trained on general concepts, yet lack a deep understanding of clinical contexts, guidelines, textbooks, publications, ontologies, and medical reasoning, which often results in inaccuracies and can have severe consequences such as misdiagnosis and/or inappropriate treatments.

To address these challenges and others, the techniques disclosed herein enable generation of targeted clinical handoff summaries for use by healthcare providers in a variety of handoff settings. The techniques described herein also provide a succinct clinical contextual summary presenting the patient's needs and status in a focused, curated manner with narrative and discrete details. The summaries include narrative and discrete detail to enable the recipient to quickly understand a given patient's status, with additional information readily accessible as needed. The summary can include what happened since the last time a physician cared for a patient (i.e. a summary of things that have changed) and/or a summary of what happened since the patient was admitted.

The techniques described herein provide innovative a routing model for selecting processing paths for generating clinical summaries. The routing model takes into consideration patient status and enables quick and efficient generation of clinical summaries particularly for use by healthcare providers in ensuring continuity of care for a particular patient over the course of a hospital admission. Given a query received from a healthcare provider with a given role, the routing model determines the appropriate set of processing modules that should be used to process EHR data related to the patient for a given category of the query. The system then generates a narrative summary and a structured summary to provide the healthcare provider with a summary of clinical information specific to the patient, the category of query, and, optionally, role of the healthcare provider.

It is noted that, the term “healthcare provider” as used herein generally refers to healthcare practitioners and professionals including, and not limited to: physicians (e.g., general practitioners, specialists, surgeons, etc.); nurse professionals (e.g., nurse practitioners, physician assistants, nursing staff, registered nurses, licensed practical nurses, etc.); and other professionals (e.g., pharmacists, therapists, technicians, technologists, pathologists, dietitians, nutritionists, emergency medical technicians, psychiatrists, psychologists, counselors, dentists, orthodontists, hygienists, etc.).

### Clinical Handoff Summary Generation System

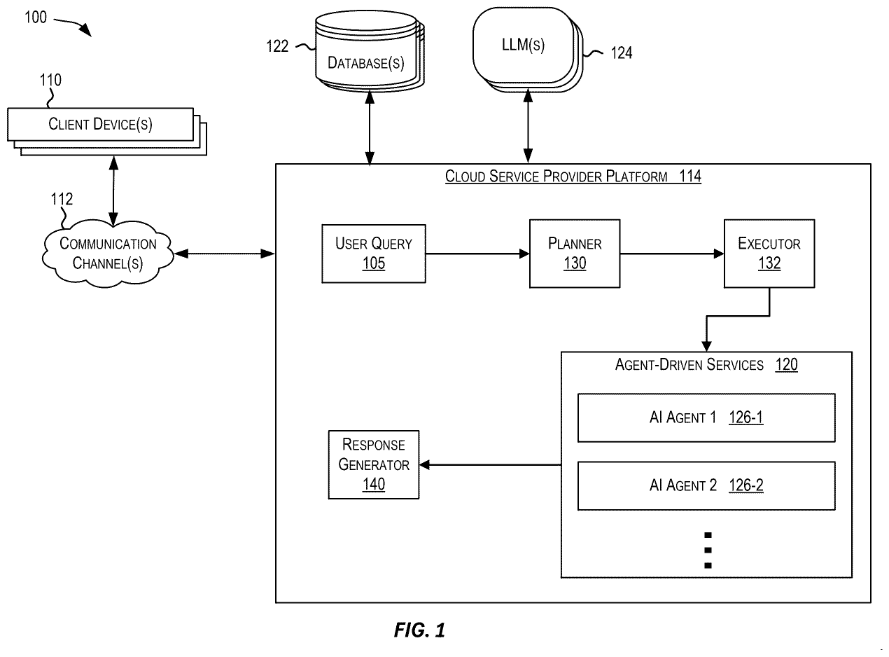

A simplified example of an agentic AI approach to receiving user queries from client devices, extracting data from multiple databases, processing the user queries by managing multiple agent-driven services, then producing a result is illustrated in FIG. 1. FIG. 1 is a block diagram illustrating an example computing environment incorporating agent-driven services, according to certain environments. In examples, the agent-driven services may include one or more artificial intelligence resources acting as “agents,” each performing a defined task or set of tasks. For instance, one of the agents may implement the summary of patient-specific information, as described herein. In an embodiment, computing environment 100 includes one or more client devices 110 (hereinafter “client devices 110”), one or more communication channels 112 (hereinafter “communication channels 112”), a cloud services provider platform 114 (hereinafter “platform 114”) including agent-driven services 120 and connected with one or more databases 122 (hereinafter “databases 122”) and one or more large language models 124 (hereinafter “LLMs 124”).

As shown in FIG. 1, agent-driven services 120 may include, for example, one or more artificial intelligence agents (hereinafter “AI agent 126”). In an embodiment, agent driven services 120 includes a plurality of AI agents, shown as AI Agent 1 (126-1), AI Agent 2 (126-2), and so on, as indicated by ellipsis. Each AI agent may be configured to specialize in a particular task, such as the modular summary generation disclosed herein. In examples, an AI Agent may call one or more of LLMs 124 to generate an execution plan (e.g., instructions), then execute the execution plan. In embodiments, an agent may itself include a model, such as an LLM, Linear Mixed Model (LMM), Small Language Model (SLM), Medium Language Model (MLM), or others.

Cloud services provider platform 114 receives user query 105 from one of client devices 110 via communication channels 112, and user query 105 is passed to a planner 130. In embodiments, planner 130 determines the appropriate course of action (e.g., selection of the appropriate AI agent with agent-driven services 120, timing and/or prioritization of tasks to be performed in response to the user query, etc.), then the action so determined is sent to executor 132.

In embodiments, at executor 132, a new execution plan may be generated or an existing plan selected out of a library of execution plans (not shown). An execution plan may include, for example, information regarding the course of action, timing, prioritization, etc. The execution plan is then performed by one or more of the AI agents within agent-driven services 120. The one or more of the AI agents performs the appropriate tasks, based on the information accessible at databases 122 and LLMs 124, to send the resulting output to a response generator 140. Response generator 140 generates then transmits a response to the client device that originated the user query 105.

While the present disclosure mentions the use of LLMs as an example mechanism for analyzing data and generating patient information summaries, it is noted that other generative artificial intelligence techniques, including other generative machine learning models may be used. Examples of such techniques and models include, and are not limited to, Small Language Models (SLMs), Multi-modal Models, reasoning models and chain-of-thought architectures, transformer-based models, and the like.

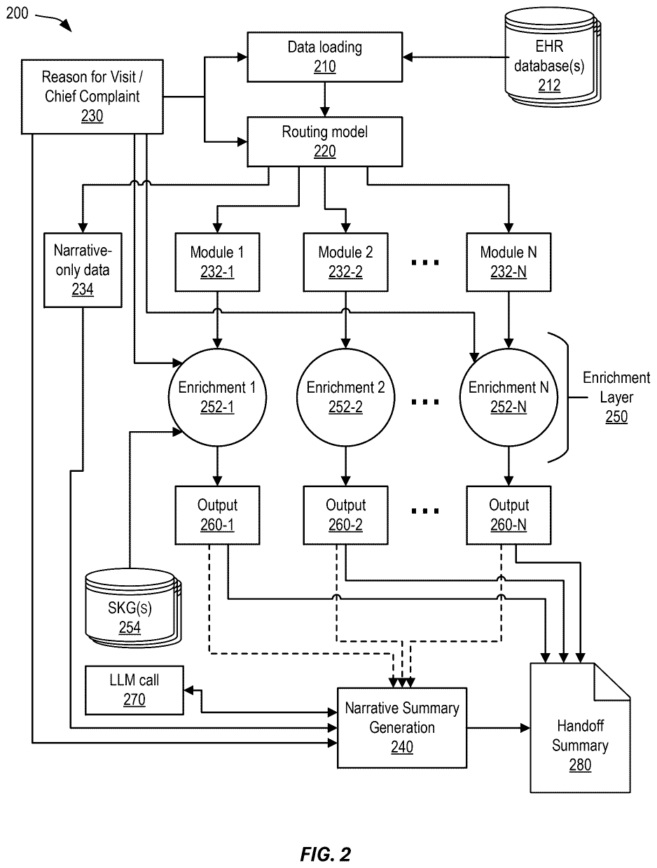

FIG. 2 is a block diagram illustrating an example of the function of portions of the cloud service provider platform of FIG. 1 in producing a clinical handoff summary, in accordance with embodiments. As shown in FIG. 2, a system 200 includes a data loading block 210 and a routing model 220. In response to a query (e.g., user query 105 of FIG. 1), data loading block 210 functions to pull, filter, and process data from one or more EHR databases 212 (e.g., database(s) 122 of FIG. 1). The query may originate from a healthcare professional (e.g., from a nurse headed off-duty, a nurse coming into a shift, an on-call physician making patient rounds, etc.). Alternatively, the query may have been automatically triggered at specified times, such as one hour prior to a scheduled shift change.

Data loading block 210 is configured to pull data and semantic objects from EHR databases 212 for a specific patient (e.g., based on a patent identification number) in consideration of reason for visit (RFV) and/or chief complaint (CC) (indicated as box 230) for the specific patient as included in the query and likely necessary for inclusion in the clinical handoff summary, as an EHR system generally includes many semantic objects, many of which are not relevant to the specific patient nor the condition(s) for which the patient is being admitted. At this initial data pull, data loading block 210 may pull a range of data, including potentially extraneous data that, while related to the patient, RFV, and CC, may not be used in the summary generation. For instance, data loading block 210 retrieves patient-specific information from the EHR database including, and not limited to:
Name
Age
Gender
Most recent vitals (height, weight, heart rate, blood pressure, etc.)
Known conditions (e.g., for a specified time frame, such as since admittance or in the past X hours, as related to the RFV/CC)
Current and past prescriptions and medications (e.g., for a specified time frame, such as since admittance or in the past X hours, and upcoming medication administration schedule).
Known allergies
New related diagnostic study data and lab results (e.g., for a specified time frame, such as since admittance or in the past X hours)
New related procedures (e.g., for a specified time frame, such as since admittance or in the past X hours, and any upcoming planned procedure).
Related social history (e.g., patient demeanor, family caregivers assisting the patient)
Admission Notes (e.g., circumstances of the admission, fall risk, behavior, any other special considerations that may help another healthcare provider on the patient team better assist the patient).

Once the relevant data has been extracted, routing model 220 determines which of the processing modules (i.e., AI agents 126 in FIG. 1) should be selected for further processing the extracted data. In embodiments, routing model 220 selects one of several predefined categories, for which a set of modules have been preselected or may be dynamically selected. The categories may include, for example, “New Admit,” “New to Me,” and “Rounded on Before.”

As an example of a routing model for determining the applicable set of processing modules, the inputs and features considered may include, and are not limited to the following:

| | |
|---|---|
| • | role: enum{nurse,physician,pharmacist,therapist} |
| • | category_hint: enum{NewAdmit,NewToMe,RoundedOnBefore}\|null |
| • | rfv, cc: strings |
| • | time_since_admission_hours: number |
| • | time_since_last_handoff_hours: number\|null |
| • | recent_events_count: {labs_abnormal:int, med_changes:int, procedures:int} |
| • | pediatric_flag: boolean |
| • | device_presence: boolean (tubes/lines/drains) |
| • | token_budget: int (LLM context window planning). |

The determination of a specific category may be performed in a rule-based manner. In examples, the determination may be made by a machine learning model trained on a training set including variations of the inputs listed above. An example determination process may be as follows:

| | |
|---|---|
| • | IF time_since_admission_hours <= 6 THEN category = NewAdmit |
| • | ELSE IF time_since_last_handoff_hours != null AND |
| | time_since_last_handoff_hours <= 12 THEN category = RoundedOnBefore |
| • | ELSE category = NewToMe |
| • | Override with category_hint if provided. |

The module selection for each category may be predicated on a variety of factors. For example, as discussed above, a set of processing modules may be preselected for each category to tailor the resulting handoff summary for each category. For instance:
NewAdmit selected modules: Problems, AdmissionNotes, Vitals, Labs, Diagnostics, Medications (home+initial orders), Procedures (initial), Social, Devices (if any)
NewToMe selected modules: IntervalHistory (notes-derived), Problems (relevancy-weighted), Vitals trend (3 to 7 days), Labs trend, Inpatient Meds (changes), Procedures (since admission), Tasks, Devices, Intake/Output (IO)
RoundedOnBefore selected modules: Changes since last_handoff_ts: med_changes, abnormal_vitals, new labs/procedures, Tasks due next shift, Devices maintenance due

It is also recognized herein that, for artificial intelligence processing, the budgeting of the units of data processed by AI models (i.e., tokens) as well as filtering of the intermediate representation (IR) of the data retrieved by data loading 210 may be considered for each category (or role, as discussed below) to reduce latency and improve efficiency of the summary generation across the cloud computing platform. Token budgeting and IR filtering may include, for example, computation of a token_cost_estimate(IR) by summing the per-entity token costs.

In examples, a process such as the below may be implemented:

| | |
|---|---|
| • | If token_cost_estimate > token_budget: |

| | |
|---|---|
| • | Apply priority order: Alerts > Problems.relevant > Medications.inpatient |

(changes) > Vitals/Labs (abnormals and trends) > Procedures (new) > Devices due >
Notes.insights > Others

| | |
|---|---|
| • | Truncate low-priority entities and retain provenance pointers for drill- |

down links.

The token budgeting process may also include fallbacks in case the category determination described above faces known challenges such as, for example,
Sparse data (i.e., insufficient data pulled from EHR databases to make a category determination for the present user query): backfill from last encounter within look-back window (e.g., 90 days) with “staleness” flags.
Conflicting category features: default to NewToMe.

For instance, a handoff summary generated under the ‘New Admit’ category summarizes known information at the time of admission, to be used by other hospital healthcare providers to quickly understand the circumstances of the admission (e.g., Emergency Room visit, specialist referral, post-surgical admittance, etc.) and any special considerations regarding the patient or the condition being treated. Thus, a “New Admit” handoff summary would specifically engage particular processing modules (e.g., selected from a plurality of modules such as module 1 (232-1), module 2 (232-2) through module N (232-N) as indicated by ellipsis in FIG. 2) relevant to producing the New Admit handoff summary. Each one of the modules 232 further extracts module-specific data (e.g., normalized to RxNorm standard identifiers for medications, Logical Observation Identifiers, Names and Codes (LOINC) terminology standard for labs, SNOMED CT or ICD-10 clinical terminology standard for problems) that are relevant to performing the specific function of that module. For instance, if module 1 (232-1) is configured for processing information related to medications, then module 1 may select the medication-related information (e.g., current and past prescribed medication, known over-the-counter medications used by the patient, medications used immediately prior to admittance, medications being used to treat the current RFV/CC, planned medication administration schedule, etc.) out of the extracted data pulled by data loading 210.

The category “New to Me” may initialize a different set of processing modules relevant for producing a clinical handoff summary suitable for a healthcare professional who is caring for a particular admitted patient for the first time. For example, the New to Me handoff summary may include more details about the medical events, procedures, administered medications, and condition changes since admission of the patient, optionally giving more weight to any events that may have occurred during the previous shift (or other specified time period, such as the past four hours). Additionally, the New to Me handoff summary may emphasize the intangible factors that may facilitate caregiving for the specific patient, such as any language barriers, known behavior issues, dynamics amongst family members who regularly visit the patient, food/lighting/television/sound preferences, and knowledge of other predilections that may allow the healthcare provider to more readily earn the trust of the patient.

The category “Rounded on Before” initializes a still different set of processing modules useful in producing a clinical handoff summary for an incoming healthcare professional who has previously cared for the particular patient during the same hospital stay. For instance, the Rounded on Before clinical handoff summary may place an emphasis on any new procedures, diagnostics, and progress since the last time the healthcare provider interacted with the patient, as well as upcoming planned procedures and medications as well as requirements and milestones to be met prior to discharge.

As a further example, the selection of processing modules may be dependent upon a defined role of the user originating the query (e.g., user query 105). For instance, a combination of processing modules and/or specific processing settings within a given processing module (e.g., selection of one or more semantic knowledge graphs or enabled type(s) of enrichments as described below), may be based on the level of permissions granted to the role. A user may be associated with one or more roles including, and not limited to, a nurse, a nurse's aide, a physician, a physician assistant, a supervising nurse, a supervising physician, a pharmacist, and others as defined within the present system as disclosed.

Identification of the user's role within the user profile allows further tailoring of the generated handoff summary. That is, the handoff summary generated for a first user in a first role may differ from the handoff summary generated for a second user in a second role. For example, the triggering of the system of the present disclosure by a nurse going off a shift may cause the system to select a set of nurse-focused operational processing modules, emphasizing tasks, devices (e.g., lines, tubes, and drains that require servicing), intake/output over the past 24 hours, and others. If the user query was initiated by an incoming on-call physician may initiate a different set of processing modules emphasizing data useful for a physician such as, and not limited to, reason for visit, chief complaint, prioritized problem history, diagnostic interpretations, procedures performed, changes since admission, pending consults, imaging, lab results, and prescribed and administer medications. A pharmacist-initiated user query may emphasize details related to the patient's medication history, prioritizing interaction alerts and contraindications for specific medications.

Alternatively or additionally to the modules 232, routing model 220 may select a narrative-only data block 234 to process at least a portion of the extracted data from the data loading. For instance, narrative-only data block 234 may selectively process only unstructured, freeform data, such as notes manually entered by previous healthcare providers that have interacted with the patient. The processing performed by narrative-only data block 234 may include, for example, optical character recognition and/or image recognition (for scanned notes) techniques for recognizing text in the unstructured data, chunking of the unstructured data by the identified information (e.g., medication, patient history, etc.), and other types of processing suitable for unstructured, freeform data entry. The unstructured data processed by narrative-only data block 234 may be passed directly to a narrative summary generation block 240, as shown in the example illustrated in FIG. 2.

In embodiments, each module 232 passes the processed data through an enrichment layer 250, which may include enrichment blocks 252 (e.g., enrichment 1 (252-1), enrichment 2 (252-2), through enrichment N (252-N), as indicated by ellipsis) for filtering, prioritizing, grouping, and otherwise refining the retrieved data, in accordance with the RFV, CC, and other information specific to the patient. For instance, module 1 (232-1) may provide selected medication-related data to enrichment block 252-1, which takes into consideration the RFV and CC in further filtering the received data. Enrichment 252-1 may also use one or more semantic knowledge graphs (SKGs) provided by SKG(s) 254, which includes information regarding relationships between the different semantic objects within medication-related data. As an example, SKG(s) 254 may recognize potential side effects, adverse contra-indications, or potential allergy issues related to a given medication, to be included in the consideration provided at enrichment 252-1. Similarly, the SKG may be used to break down data into groups such as “relevant,” “other ongoing,” and “irrelevant” according to the relevancy of the data to a chief complaint or reason for visit, for example.

Each enrichment block produces an output 260 (e.g., output 1 (260-1), output 2 (260-2), through output N (260-N), as indicated by ellipsis), which may include intermediate representation (IR) of the data processed through module 1 and enrichment 1. In embodiments, each output includes the processed data in IR format, with a standard syntax suitable for submission to a Large Language Model (LLM) and/or artificial intelligence (AI) layer. For example, the processed data in IR format has been transformed to have a structured, computer-interpretable encoding format, such as those commonly used in natural language processing.

An example of the logic used in enrichment and SKG-driven prioritization in the example of the generation of a handoff summary may be as follows. Such an enrichment process using one or more SKGs is particularly useful following normalization of the extracted data into the standard IR format, which enables the SKG to prioritize and filter discrete data based on relevance to the reason for visit and/or chief complaint.

SKG structure includes parameter definitions such as, for example:

| | |
|---|---|
| • | Nodes: {Condition(SNOMED/ICD10), Symptom, Lab(LOINC), |

Medication(RxNorm), Procedure(CPT/SNOMED), Allergy, Device}

| | |
|---|---|
| • | Edges: {treats, contraindicated_with, indicates, side_effect_of, |

requires_monitoring, is_child_of (ontology), temporal_precedes}

| | |
|---|---|
| • | Versioning: skg_version: semver; updated_at: datetime; source_ontologies: |

[SNOMED, RxNorm, LOINC, ICD-10]

As an example, a relevance scoring (e.g., per problem or condition) method may be used using a calculation such as the following:

| | |
|---|---|
| • | relevance_score = w1recent_event_weight + w2severity_weight + |

w3med_interaction_weight + w4trend_weight + w5*category_alignment

| | |
|---|---|
| • | recent_event_weight: 1 if related event in window; else 0 |
| • | severity_weight: map to {critical=3, high=2, medium=1, low=0} |
| • | med_interaction_weight: 1 if SKG shows contraindication/interaction with |

active meds; else 0

| | |
|---|---|
| • | trend_weight: 1 if associated labs/vitals trending adverse per thresholds; |

else 0

| | |
|---|---|
| • | category_alignment: 1 if node is parent/child of CC/RFV in SKG; else 0 |

| | |
|---|---|
| • | Thresholds: relevant if score ≥ 3; otherwise “other ongoing” |

Trend detection may be performed on the extracted data using methods such as, for example:
Z-score method: For labs with ≥3 values in window, flag abnormal if |(latest-mean)/std|≥2 and direction matches adverse clinical direction (consult SKG “adverse_direction” attribute).
Exponentially Weighted Moving Average (EWMA) for vitals: EWMA_t=α*value_t+(1−α)*EWMA_(t−1); flag if EWMA crosses age/weight-adjusted bounds.
Delta-from-baseline: baseline=median over prior 7 days or pre-admit; flag if abs (latest-baseline)≥configured delta.

The data processing may also consider adjustments to the analysis based on the age of the patient. For example, for a pediatric patient, adjustments such as the following may be considered:
Use age/weight-indexed normal ranges from Pediatrics reference tables (in SKG attributes).
Convert all doses to mg/kg where applicable; flag out-of-range dosing.

Deduplication and conflict resolution in the extracted data may include setting parameters such as the following:
Notes: compute duplicate_hash over normalized text (strip headers/signatures); drop duplicates within 24 h unless author differs and sections add net-new insights.
Labs/Vitals: unify units, convert to canonical; if conflicting timestamps within +2 minutes, prefer analyzer with higher trust score (configured per device/lab source).
Meds: collapse identical orders differing only by administration route comments; keep most recent active.

The enrichment and prioritization processes may further include generation of alerts in the handoff summary and/or user interface using logic and rules such as the following:
Med contraindication: if SKG has contraindicated_with edge between active Med and Problem/Allergy→severity: critical.
Lab outlier with trend: if latest abnormal AND trend adverse→severity: high.
Device maintenance due within next shift window (≤8 h)→severity: warning.

In an example, each alert may include information such as a message, linked_entities (IR references), and recommended next action (e.g., “verify order,” “notify attending”).

In order to ensure provenance and narrative grounding in the produced handoff summary, various mitigation measures may be included such as, and not limited to the following:
Every narrative sentence is generated from one or more IR references; include sentence-level provenance map: sentence_id→[IR_entity_ids].
UI enables hover/click to show source system, record IDs, timestamps, and author.

Caching and/or invalidation of the extracted data may be performed based on preset logical rules such as, for example:
Cache key: {patient_id, encounter_id, category, window_from_ts, window_to_ts, skg_version}
Time to Live (TTL): default 30 minutes; event-driven invalidation on: new lab result, med order change, device event, note added.
Concurrency: optimistic locking by IR version; merge deltas with conflict resolution rules above.

Continuing to refer to FIG. 2, outputs 260-1, 260-2, etc. may optionally be provided to narrative summary generation 240, which engages an LLM call 270 to generate a narrative summary section of a clinical handoff summary 280. Narrative summary generation 240 may take into consideration information regarding the RFV/CC, processed data from narrative-only data 234, as well as, potentially, outputs from the enrichment layer in generating a narrative summary section of the clinical handoff summary 280.

LLM call 270 is used to generate a collection of phrases and sentences that may be used a part of the narrative summary section. LLM call 270 may also include a repository of commonly used prompts and context statements suitable for clinical handoff situation, such as, “As a nurse, I want a summary of all relevant patient information, emphasizing events during the past four hours, so that I can use the summary to hand off my patient to the new incoming nurse during a shift change.”

The collection of phrases and sentences are presented to narrative summary generation 240, which generates the contents of a narrative portion of a handoff summary 262. For example, narrative summary generation 240 may process the data presented therewith through natural language processing methods to generate suitable components of a narrative summary based on the extracted information. In examples, narrative summary provides an informative summary of patient information, particularly highlighting the relevant conditions as relevant to the specific patient, RFV, and CC.

Narrative summary generation 240 may generate a standalone report in narrative format and/or combined with outputs 260 into a structured format. In embodiments, narrative-only data 234, outputs 260, and the output from narrative summary generation 240 may be stored in memory at the cloud service provider platform (e.g., cloud service provider platform 114 of FIG. 1) until specifically requested by the user. Particularly if the initial query was automatically triggered by the cloud service provider platform at a shift change time, then there is a possibility that the query may have triggered functionalities of the cloud service provider platform, such as other agentic AI processes. In such a case, planner 130 of cloud service provider platform may provide prioritization instructions to the various agent-driven services to present any outputs from the AI agents in an order appropriate for a specific user case scenario.

Clinical handoff summary 280 further includes a structured section, including outputs 260-1, 260-2, etc. formatted into a predetermined layout. The structured section may include, for example, a graph showing the patient's latest vital signs, most recent medication administration details and the next scheduled administration time, insertion/cleaning time of any tubes or lines, and other numerical and structured information, presented in a predictable way.

For instance, the structured section lists data that is relevant to the current patient. The narrative section supplements the structured data with important qualitative information that cannot be gleaned from the structured data alone, such as data from notes or a relationship in the data.

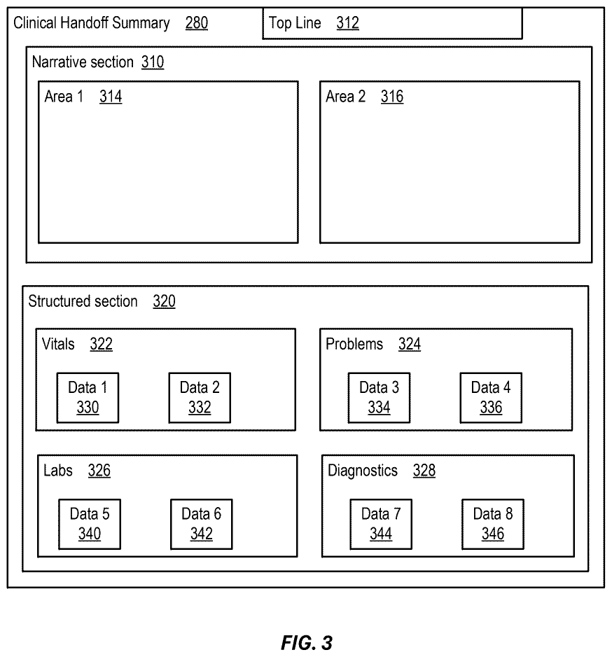

FIG. 3 is an example layout of a clinical handoff summary, in accordance with embodiments. As shown in FIG. 3, in accordance with embodiments, clinical handoff summary 280 includes a narrative section 310 (including, as an example, the output from narrative summary generation 240 of FIG. 2) and a top line 312, which includes patient identifying information, such as patient name and room number. Narrative section 310 may include one or more areas (shown as area 1 (314) and area 2 (316)), into which narrative related to specific topics may be inserted. For instance, area 1 (314) may be used to present a general summary of the patient's personal information, RFV/CC, and any related history. Area 2 (316) may be reserved for a summary of recent procedures, known allergies, and other information that may be drawn from extracted unstructured data.

The information presented in narrative section 310 may be an extract from the narrative summary generated by narrative summary generation 240 and/or independently populated using outputs 260 of FIG. 2. In embodiments, narrative section 310 may include a generated summary of the relevant information in natural language format, bullet points of key information, extracts from notes from previous patient encounters, electronic scans of historical notes, and other long-form information.

Clinical handoff summary 280 may additionally include a structured section 320. In the illustrative example, structured section 320 may include one or more areas (shown as vitals 322, problems 324, labs 326, and diagnostics 328, as an example), in which structured data such as lab test results and lists of known allergies and conditions may be presented (represented as data 1 (330), data 2 (332), data 3 (334), data 4 (336), data 5 (340), data 6 (342), data 7 (344), and data 8 (346) in FIG. 3). The different areas may include structured data extracted from outputs 260 used to populate a predetermined template, such as in the form of a graph, a list, and other presentations.

Optionally, uniform resource locators (URLs) linking to additional information may also be embedded within keywords and phrases within narrative section 310. That is, rather than presenting all relevant information at once on the screen, certain keywords and/or phrases may be embedded with URL links to information located on the EHR or elsewhere. In this way, the amount of data presented to the user on a screen may be limited, even with a thorough narrative summary, while allowing the option of retrieving more detailed information via the URL links. Such visual representation of structured data may assist the healthcare provider in identifying key information required to provide the appropriate level of care for a specific patient.

In certain embodiments, clinical handoff summary 280 may include a search field or a user interface “button” to allow the healthcare provider to regenerate the handoff summary based on any newly added information (e.g., a newly performed procedure such as intubation, new condition identified, etc.) and the previously extracted data at data loading 210. If necessary, additional information may be pulled by data loading 210. In certain embodiments, the healthcare provider may be presented with an option to save the current summary report to a file folder in memory, prior to generating an updated summary report. For instance, as a particular patient may present with multiple medical conditions, additional and/or updated summary reports may be necessary for a complete assessment of the patient history. As another example, certain portions of the unstructured section may be enhanced with structured information, such as URL links to access external documents (e.g., images of handwritten notes).

In this way, clinical handoff summary 280 enables healthcare providers to obtain a trove of information regarding a patient as related to a specific patient's current and previous medical history particularly related to one of the categories of New Admit, New to Me, or Rounded on Before, in a compact format, suitable for display on a small screen such as a tablet, as well as links to additional information of so desired. In embodiments, one or more portions of clinical handoff summary 280 may be reserved to allow the healthcare provider to enter additional notes.

As a specific example, the various sections of the clinical handoff summary 280 may include information such as the following:

### Narrative Section:

1. Top sentence including: full name, preferred nickname, age and gender, reason for hospitalization, patient highlights (e.g., most significant changes in condition since admittance);
2. Patient location (optional): hospital name, unit name, room number;
3. Admission history: Chief compliant, reason for visit, reason for admission with diagnosis, admission date and time, previous unit transfer history;
4. History of illness (particularly important for New Admit and New to Me): High-level review of the history of present illness (HPI) relevant to the present admission;
5. Interval history: A summary of progress/change of past 24 hours (or some other set period), progress of illness; progress of any previously noted abnormality, night events, abnormal vitals, change of medications, new medication orders;
6. Intake course/pre-hospital course (for New admit): Important treatment, diagnostics, procedures and conditions (such as noted in an Emergency Department intake) before admission, important upcoming procedures or treatment plan, dates for procedures and diagnostics, listed in chronological order;
7. Hospital course (for New to Me and Rounded on Before): Important previous treatment, diagnostics, procedures and progress of illness during hospitalization, important upcoming procedures or treatment plan, discharge plan;
8. New progress since last seen (for Rounded on Before): New procedures, diagnostics, conditions and progress since last seen, discharge plan.

### Structured Section:

1. Vital signs: Show last 24 hours trending, or more days for New to Me, showing a range per day (e.g., minimum and maximum) per day if there are multiple values for a given time period, highlight abnormal values and positive cultures, extended trends of longer time for specific vitals, a line graph for showing the past few days (3-7 days) trending, a vitals list sorted in a consistent order;
2. Labs: Results sorted as groups as stored in the EHR, highlight abnormal values and positive cultures, showing a range per day (e.g., minimum and maximum) per day if there are multiple values for a given time period, include extended trends of longer time for specific labs, show relevant labs with extended link to show the full list, the lab results after the patient initially visited the hospital for the present condition, the lab results of the most recent values of last visit (outpatient or inpatient);
3. Problems: broken down into ‘relevant’ and ‘other ongoing,’ sorted by relevance using SKG, common and/or non-conflicting issues, problems for the present admission, new problems since admission or last seen by a healthcare provider;
4. Diagnostics: summaries of multiple reports, if any, links to view each full report and imaging, sorted by chronological order, diagnostics during hospital stay
5. Home medications: medication name, sorted by alphabetical order, dosing and frequency, relevancy;
6. Hospital medications: broken down into scheduled, pro re nata (PRN), and intravenous (IV), sorted by alphabetical order, dosing, frequency and start time;
7. Procedures: performed during the present visit, past relevant procedures within a look-back period (e.g., 1 to 3 years);
8. Notes: Intake physician note (including normalized section headers, e.g., HPI, assessment and plan (A&P), medical decision making (MDM), history of present illness (HPI), recommendations, transfer note, discharge summary of recent clinical encounters;
9. Social history: e.g., smoking and/or alcohol consumption;
10. Intakes/outputs: food/nutrition intake in past 24 hours, urine output, stooling and pain scales, I/O net balance (e.g., fluid balance);
11. Immunizations: e.g., indication of overdue immunization;
12. Special considerations for pediatric patients: All metrics (meds, labs, vitals, etc.) calculated as weight-based, lab results flagged based on age and weight (normal ranges are based on age and weight), nutrition/feeding plan (for babies), diaper information, growth chart, known assessments.

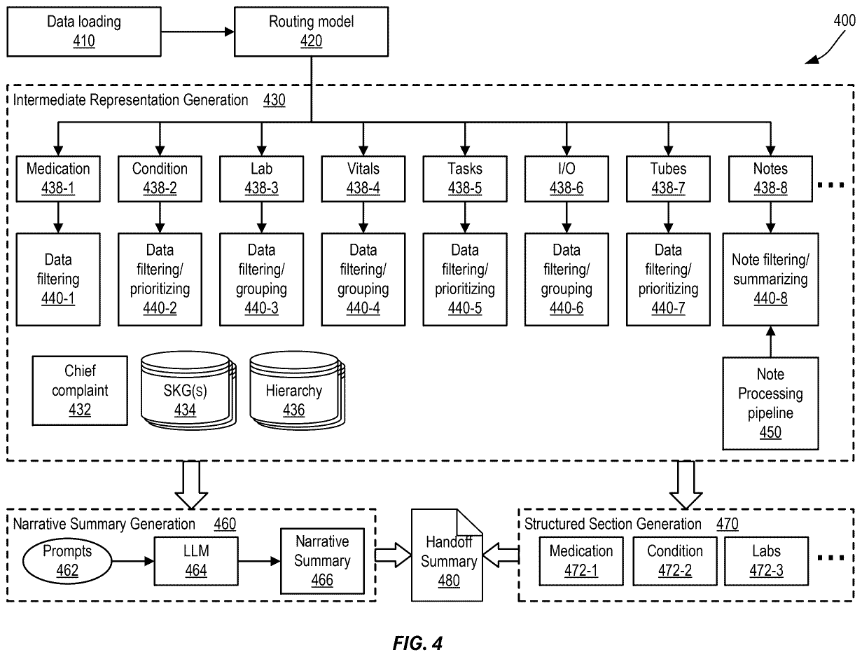

FIG. 4 is a block diagram illustrating an alternative example of the function of portions of the cloud service provider platform of FIG. 1 in producing a clinical handoff summary, in accordance with embodiments. Similar to system 200 of FIG. 2, a system 400 includes a data loading block 410 from an EHR in response to a query (such as database(s) 122 and user query 105 of FIG. 1). The data pulled by data loading block 410 is directed to routing model 420 (e.g., routing model 220 of FIG. 2), which determines a category relevant to the query initiating the functions of system 400. The categories may include, for example, the New Admit, New to Me, and Rounded on Before as described above. Accordingly, routing model 420 directs the relevant data to an intermediate representation (IR) generation block 430.

In the example illustrated in FIG. 4, IR generation 430 includes information regarding a repository of chief complaints 432 for use in segregating relevant data into more refined segments based on the specific chief complaint in question. IR generation 430 also includes one or more SKGs 434, including relationships between disparate pieces of semantic objects within the relevant data. IR generation further includes a hierarchy database, including predetermined priority orders between different semantic objects considered.

Routing model 420 distributes the relevant data to one or more processing modules (i.e., equivalent to modules 232 in FIG. 2), in accordance with the category relevant to the query. For instance, IR generation 430 may include a plurality of processing modules 438 dedicated to processing related to medication 438-1, condition 438-2, lab results 438-3, vitals 438-4, tasks 438-5, intake/outputs (I/O) 438-6, tubes/lines/drains 438-7, notes 438-8, and more (as indicated by ellipsis). Medication module 438-1 may be specialized to process data related to the patient's medication history, medications administered during the current admittance, upcoming schedule of planned dosages and administration times, previously administered medications in other settings, existing and previous prescriptions, known reactions to previously administered medications, and similar such information for the patient. Condition module 438-2 may process information specific to the condition for which the patient is being treated during the present hospital stay. Lab module 438-3 may be configured to extract and process information related to recent and past lab results as related to the current chief complaint. Vitals module 438-4 tracks the latest and past vitals measurements of the patient taken during the current admittance, comparisons with baseline values as collected during other clinical visits with the patient, and similar information. Tasks module 438-5 may, for example, pull information relation to scheduled tasks (e.g., medication administration, wound dressing change, physician/surgeon follow-ups). I/O module 438-6 may perform specialized processing related to food or nutrition intake and outputs (e.g., stool, urine). Tubes/lines/drains module 438-7 may process data related to intubation, central line management, drain cleaning, and other information such as when tubes/lines/drains were installed and when they need to be changed. Notes module 438-8 may process unstructured data such as notes entered into the EHR in freeform, previous manual notes, and/or handwritten notes by healthcare providers.

One or more of processing modules 438 may be selected by routing model 420 in accordance with the specific category of the handoff summary being generated. For example, in a Rounded on Before handoff summary may not require as much information regarding condition 438-2 as the healthcare provider is already familiar with the condition for which the patient has been admitted. Instead, a Rounded on Before handoff summary may emphasis any changes to the patient or medical events that may have occurred since the last time the healthcare provider interacted with the patient.

Each of processing modules 438 provides its respective processed data to an enrichment block 440 (e.g., enrichment layer 250 of FIG. 2). For instance, processed data from medication module 438-1 may be further enhanced by data filtering provided at enrichment block 440-1. Enrichment block 440-2 affiliated with condition module 438-2 may further include data prioritizing functions to ensure the most important information related to the condition is surfaced in the subsequently generated summary documents. Enrichment block 440-3 receiving data from lab module 438-3 may further group data to be presented according to type, significant changes in values, and other criteria. Notes module 438-8 may pass the processed data to enrichment 440-8, which may provide note filtering and/or summarizing functions, such as provided by an additional note processing pipeline such as character recognition, chunking operations, and de-duplication of templated text and signature blocks, for example. In embodiments, each of the processing modules and associated enrichments are configured to produce outputs in IR format suitable for further processing. Optionally, each enrichment may be further configured to identify trends in the processed data, to create alerts if certain data may be outside of acceptable normal range (or a preset threshold). Such alerts may enable even less experienced healthcare providers to quickly identify potentially concerning data and seek guidance from more experienced clinicians as necessary.

At least a portion of the processed and enriched data may be provided to a narrative summary generation 460. Like narrative summary generation 240 of FIG. 2, narrative summary generation 460 may include generation or retrieval of prompts 462 to be fed into one or more LLMs 464 in order to generate a narrative summary 466. Similarly, portions of the processed and enriched data may be provided to a structured section generation 470, which receives and formats the received data into predefined templates, graphs, lists, and other representations suitable for inclusion in a summary. Finally, outputs from narrative summary generation 460 and structured section generation 470 are consolidated into a handoff summary 480, which may have a structure such as discussed above with respect to FIG. 3.

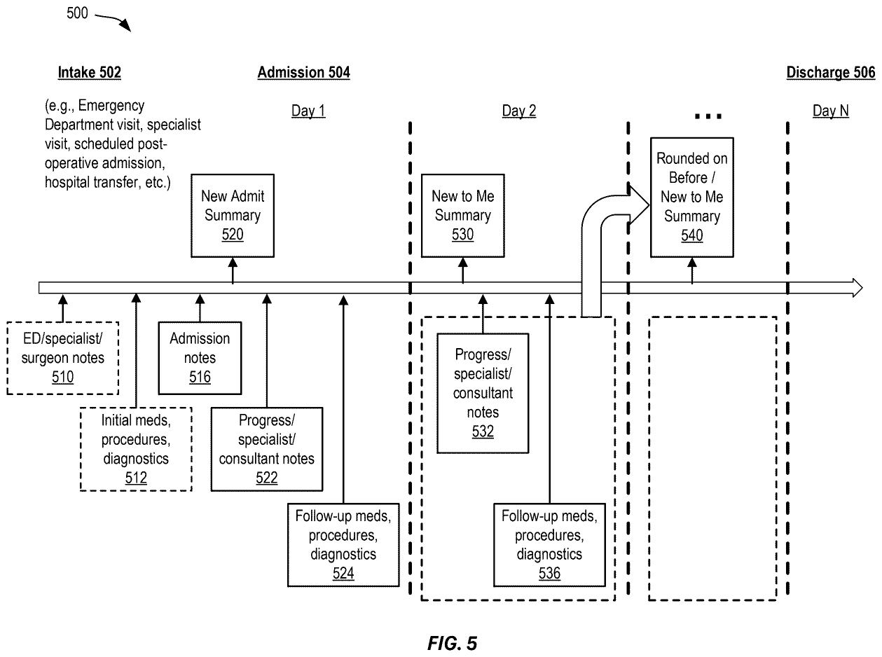

FIG. 5 is a simplified diagram illustrating an example of a timeline in producing a series of clinical handoff summaries, in accordance with embodiments. As shown in FIG. 5, a timeline 500 may generally be split into different time periods such as Intake 502, Admission 504, and Discharge 506, which may take place on the same day as the admission or N days later (shown as Day N in FIG. 5). Intake may include, for example, a visit to the Emergency Department (ED), a specialist visit, a scheduled post-operative admission, a transfer from a different hospital, and other situations.

In an example embodiment, timeline 500 may optionally include entry of ED, specialist, and/or surgeon notes 510 as well as a record of any initial medications, procedures, and/or diagnostics 512 at intake. Such information from intake may be used to generate a New Admit handoff summary 520 using system 200 or 400, for example. The New Admit handoff summary may be used at admission 504 to enable the care team upon admission to receive patient information upon admission at the hospital.

Following admission on Day 1, various progress/specialist/consultant notes may be entered into the EHR by the hospital staff. Additionally, follow-up medication, procedures, and diagnostics may be recorded during Day 1. On Day 2, or upon an initial shift change, a New to Me handoff summary 530 may be generated, consolidating data regarding the activities of Day 1 up to that point. On Day 2, and on subsequent days during the patient's hospital stay, additional New to Me and/or Rounded on Before handoff summaries may be generated upon shift changes, staff handoffs, or as needed, as progress notes and follow-up medical events occur on those days. As discussed above, the subsequent handoff summaries may apply filters, grouping, and prioritization to ensure the most important and up-to-date information is surfaced in the summary report.

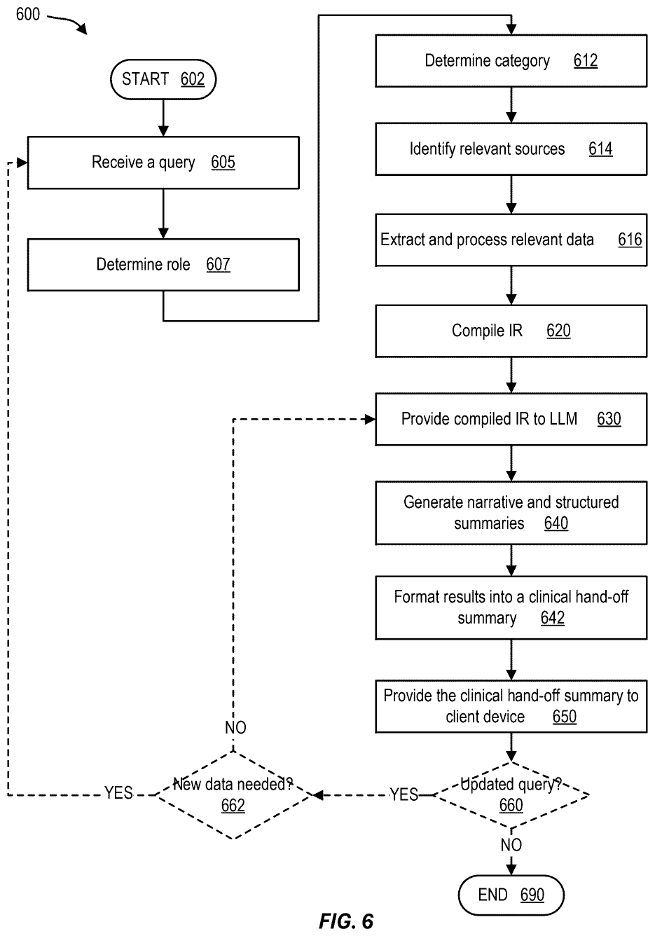

An example process suitable for use in generating clinical handoff summaries as described above is illustrated in FIG. 6, showing a flowchart illustrating a process for generating a response (i.e., a handoff report) in accordance with a user-provided query, in accordance with embodiments. A process 600 as shown in FIG. 6 may be performed in a computing environment (e.g., by cloud service provider platform 114 of FIG. 1).

Process 600 is initiated in a start step 602, then proceeds to receive a query in 605. The query may have been received from a client device (e.g., client device 110) or automatically generated by a component or service within or outside of cloud service provider platform 114. In the example process 600, a role of the query originator or purpose of the report generation query is extracted from the received query in 607. As discussed above, the initial query may be automatically triggered by the cloud service provider platform in anticipation of a scheduled shift change of healthcare providers. Alternatively, the query may specifically be originated by a user in preparation for a handoff.

The specific category (e.g., New Admit, New to Me, Rounded on Before, etc.) of the handoff summary to be created in response to the query is identified in block 612. In block 614, the process proceeds to identify the relevant sources of data from which the query-related data should be pulled, such as a part of the functionality of data loading block 210 of FIG. 2 or 410 of FIG. 4.

Process 600 proceeds to extract and process the relevant data from the EHR in block 616. The extraction may take into consideration a variety of patient and condition data, such the reason for visit, chief complaint, semantic knowledge graph, and other factors. The processed data is then compiled in block 620 as a set of intermediate representation of the retrieved semantic objects such that the IR is readily digestible by an LLM.

An example IR schema may be formatted as JSON-like, typed fields, normalized to standard terminologies, with each entity carrying provenance. In this way, the subsequent processing the data in IR format can be performed more efficiently within the cloud services platform environment.

Different types of IR included in the schema may include, and are not limited to, the following:

| | |
|---|---|
| • | PatientIR |

| | |
|---|---|
| • | patient_id: string |
| • | name: {full: string, preferred: string\|null} |
| • | demographics: {age: number, gender: string, dob: date} |
| • | location: {facility: string, unit: string, room: string} |
| • | contacts: [{name: string, relationship: string, phone: string}] |
| • | pediatric: {weight_kg: number\|null, height_cm: number\|null, |

gestational_age_wk: number|null}

| | |
|---|---|
| • | EncounterIR |

| | |
|---|---|
| • | encounter_id: string |
| • | admission_ts: datetime |
| • | category: enum{NewAdmit, NewToMe, RoundedOnBefore} |
| • | rfv: string |
| • | cc: string |
| • | last_handoff_ts: datetime\|null |
| • | time_window: {from_ts: datetime, to_ts: datetime} |

| | |
|---|---|
| • | VitalsIR (normalized units; latest and trends) |

| | |
|---|---|
| • | vitals: [{ |

type: enum{HR,BP_SYS,BP_DIA,RR,SpO2,TempC,WeightKg},
values: [{ts: datetime, value: number, unit: string],
baseline: {value: number|null, window: string|null},
flags: {abnormal: boolean, trend: enum{rising,falling,stable}|null}
}]

| | |
|---|---|
| • | LabsIR |

| | |
|---|---|
| • | panels: [{ |

loinc_code: string,
name: string,
results: [{ts: datetime, value: number|string, unit: string, ref_range:

{low:number, high:number}}],

latest_abnormal: boolean,
trend: enum{rising,falling,stable}|null
}]

| | |
|---|---|
| • | MedicationsIR |

| | |
|---|---|
| • | home_meds: [{rxnorm: string, name: string, dose: string, freq: string, |

route: string, last_fill_ts: datetime|null}]

| • | inpatient_meds: { |
|---|---|
| | scheduled: [MedOrder], prn: [MedOrder], iv: [MedOrder] |
| | } |
| • | MedOrder: { |

rxnorm: string, name: string, dose: string, freq: string, route: string,
start_ts: datetime, next_due_ts: datetime|null, last_admin_ts: datetime|null,
indications: [snomed:string]|[ ],
contraindications: [snomed:string]|[ ],
allergy_risk: enum{none,possible,likely}
}

| | |
|---|---|
| • | ProblemsIR |

| | |
|---|---|
| • | relevant: [Problem] |
| • | other_ongoing: [Problem] |
| • | Problem: {code_system: enum{SNOMED,ICD10}, code: string, name: |

string, onset_ts: datetime|null, status: enum{active,resolved}, relevance_score: number}

| | |
|---|---|
| • | ProceduresIR |

| | |
|---|---|
| • | procedures: [{code_system: enum{CPT,SNOMED}, code: string, name: |

string, ts: datetime, setting: string, relevance_score: number}]

| | |
|---|---|
| • | DiagnosticsIR |

| | |
|---|---|
| • | studies: [{type: string, ts: datetime, impression: string, link: url, abnormal: |

boolean}]

| | |
|---|---|
| • | NotesIR |

| | |
|---|---|
| • | notes: [{ |

note_id: string, author_role: string, ts: datetime,
sections: [{header:

enum{HPI,AssessmentPlan,MDM,EDCourse,Discharge}, text: string}],

| | extracted_insights: [Insight], duplicate_hash: string |
|---|---|
| | }] |
| • | Insight: {type: enum{allergy,med_change,abnormal_event}, text: string, |

linked_entities: [ref]}

| | |
|---|---|
| • | IOIR |

| | |
|---|---|
| • | intake_ml_24h: number\|null |
| • | output_ml_24h: number\|null |
| • | pain_scores: [{ts: datetime, score: number}] |

| | |
|---|---|
| • | DevicesIR (tubes/lines/drains) |

| | |
|---|---|
| • | devices: [{type: enum{ETT,CentralLine,Drain,Foley}, placed_ts: |

datetime, last_maintenance_ts: datetime, next_due_ts: datetime}]

| | |
|---|---|
| • | SocialIR |

| | |
|---|---|
| • | smoking_status: string\|null |
| • | alcohol_use: string\|null |
| • | caregivers: [{name: string, relationship: string, availability: string}] |

| | |
|---|---|
| • | AlertsIR |

| | |
|---|---|
| • | alerts: [{type: enum{lab_outlier,med_contraindication,vital_trend}, |

severity: enum{info,warning,critical}, ts: datetime, message: string, linked_entities: [ref]}]

| | |
|---|---|
| • | ProvenanceIR (e.g., attached to each element via metadata.provenance) |
| • | metadata: {source_system: string, source_record_id: string, ts_ingested: datetime, |

user_id: string|null}

The IRs as compiled are then provided to a generative resource, such as a trained LLM, in block 630, which in turn generates at least a portion of narrative and/or structured summaries in block 640. The results of block 640 may then be formatted into a handoff summary (e.g., clinical handoff summary 280 of FIG. 2) in block 642, and the handoff summary may be provided to the client device or the originator of the query in block 650. In certain embodiments, rather than providing the handoff summary to a user, the IR, narrative summary, structured summary, and/or handoff report may be stored in memory at the cloud service provider platform for future use.

Multiple approaches are contemplated for the generation of the narrative and structured summaries and are considered to be a part of the present disclosure. For example, zero-shot prompting, one-shot prompting, stepwise prompting may be considered for all or different aspects of the summary semantic object generation.

Optionally, a decision may be made in a determination 660 whether an updated or new query related to handoff has been received, such as entered at a client device user interface, based on a search performed by the healthcare provider reading the summary report, clickthroughs to URLs provided as part of the summary report, and other information. For instance, changes to the patient condition and other medical events may require generation of an updated handoff summary for the patient.

If an updated query has been received, then a determination 662 is made whether new or additional data is required to respond to the updated query. If determination 662 results in YES, new data is needed (e.g., if a new condition or medication has been identified), then process 600 returns to block 605 to process the updated query anew. If determination 662 results in NO, new data is not needed and the new query may be addressed using the extracted initial IR, the process 600 returns block 630 to process the relevant data anew in view of the updated query, thus enabling savings in computational time and resources. If no updated query has been received, then process 600 is terminated at end step 690.

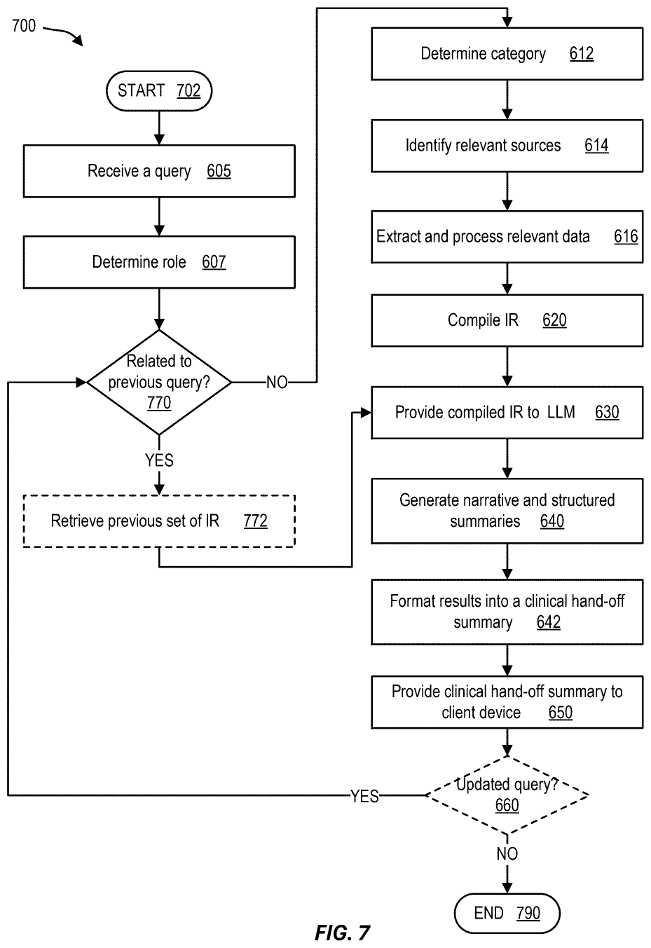

FIG. 7 is a flowchart illustrating an alternative process for generating a clinical handoff summary in accordance with a user-provided query, in accordance with embodiments. As shown in FIG. 7, a process 700 shares several of the processing blocks as shown in FIG. 6. However, upon initiating process 700 at a start step 702, the query is received in 605 and the relevant role is determined in 607, a determination 770 is made whether the received query is related to a previously presented query. If determination 770 results in NO, that the received query is unrelated to previously submitted queries, then process 700 proceeds to block 612 and follows the processing steps as shown in FIG. 7.

If determination 770 yields YES, the presently received query is related to a previous query, then process 700 optionally proceeds to retrieve a previously extracted set of IR (e.g., from cached records at the client device or within the cloud services platform) in block 772 then to provide the previous set of IR as part of the compiled IR to LLM at 630. Alternatively, for example if the previously extracted IR is still active at the cloud services platform, then process 700 may immediately proceed to block 630.

The techniques described in FIGS. 6 and 7 allows efficient production of multiple patient summaries with the most up-to-date data. In embodiments, when a summary generation is requested, then the pre-computed modules (i.e., extracted and/or filtered IRs) may be loaded to maximize reuse of semantic objects that have already been processed, then the handoff summaries may be generated using minimal computational resources (e.g., LLM tokens).

In embodiments, the clinical handoff summaries should provide the healthcare provider to focus on the following three things:
1. Clinical progression (i.e., what is the current medical plan of care);
2. Preparation plan for post-acute care (i.e., the necessary steps to get the patient ready for discharge and post-acute care);
3. Awareness of the projected length of stay (LOS).

For instance, the ISBAR (Introduction, Situation, Background, Assessment, Recommendation) framework may be followed in generating the narrative section: Situation (patient name, basic info, reason for admission), Background (allergies, transfer history, order changes, admission history (why the patient visited the ED and was admitted)), Assessment (important procedures and diagnoses, important procedures and assessments after admission, with content varying by subtype), and Recommendation (next steps for care, treatment plan, upcoming procedures, nursing care for the next shift, and discharge planning). The structure section may provide more detailed, discrete data such as problem lists, medications, procedures, labs, vitals, risk factors, upcoming activities, diagnostics, social/family history, support contacts (The patient's or the family's contact information, including family name, relationship, and phone number), intakes and outputs, clinical notes, and nursing assessments, all drawn from EHR or a semantic object database

The techniques provided herein enable significantly reduced latency, with the handoff summary generation being possible on the fly while improving the accuracy and completeness of the information included in the summary. The system prioritizes highly relevant conditions and notes, extracting and displaying only the most important information for the healthcare provider. There are also optimizations for LLM token limits, with the IR filtered to only include valuable data, ensuring efficient processing even with large amounts of patient data.

The techniques disclosed herein expedites the processing of EHR-stored data and ensures the right logic is applied to select and present relevant data. The system leverages clinical logic, such as SKG, to relate clinical conditions and prioritize them, without requiring review by a clinical expert. Both the narrative and structured section are presented, with the narrative giving a high-level overview and the structured section providing detailed, filtered data. The IR generation includes filtering to address LLM context window constraints, only including the most useful information for summary generation.

Further, the enrichment provided by the disclosed systems prioritize conditions by relevance, so the nurse sees the most important issues first. The note processing pipeline extracts and filters only the most important notes and sections. The narrative and structured sections may be coupled, providing both a high-level summary and detailed discrete data as needed. The IR includes initial filtering based on business rules to ensure only relevant information is presented to the LLM and the user interface (UI). The IR used in generating the summaries are filtered to include only the most useful pieces for the narrative and structured sections, tailored to specific situations (e.g., the categories identified above).

The developed approach described herein addresses these challenges and others by providing techniques for assisting healthcare providers with necessary and time-consuming and often tedious tasks. Techniques are disclosed herein for improving the efficiency of and reducing the computing resources required to perform various healthcare services in a clinical environment. In certain embodiments, techniques are disclosed for equipping a healthcare provider end user with a clinical software application that can be installed on and utilized from one or both of a mobile computing device and a desktop computing device to facilitate performance of the various tasks typically rendered by a healthcare provider as part of providing healthcare services to patients.

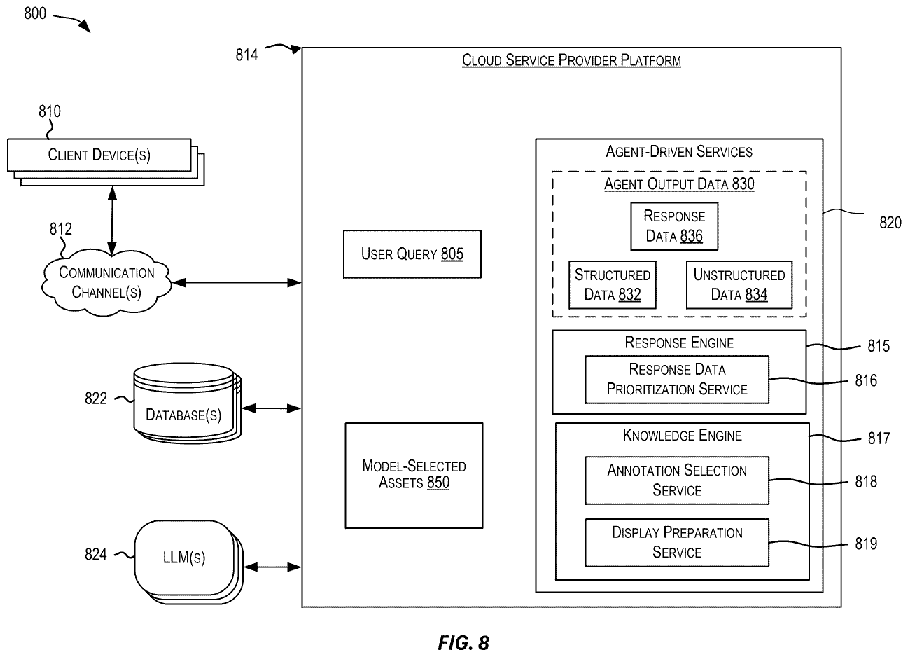

FIG. 8 shows a simplified diagram depicting a computing environment 800 incorporating an agent-driven digital assistant system configured to generate knowledge-grounded response data according to certain embodiments. As shown in FIG. 8, the computing environment 800 includes one or more client devices 810 (hereinafter “client devices 810”), one or more communication channels 812 (hereinafter “communication channels 812”), a cloud service provider platform 814 (hereinafter “platform 814”), one or more databases 822 (hereinafter “databases 822”), and one or more LLMs 824 (hereinafter “LLMs 824”). The platform 814, which can be included as part of a cloud infrastructure of a cloud service provider (e.g., Oracle Cloud Infrastructure or OCI), can be configured to communicate with, send data and information to, and receive data and information from the client devices 810 via the communication channels 812. Additionally, the platform 814 can be configured to access and/or call the databases 822 and the LLMs 824 to obtain and/or receive data and information from the databases 822 and the LLMs 824. Data and information received from the client devices 810, the databases 822, and the LLMs 824 can be used by the platform 814 to execute tasks and perform services such as automatically generating one or more portions of knowledge-grounded response data. While FIG. 8 shows the databases 822 and the LLMs 824 as being separate from the platform 814, this is not intended to be limiting, and one or more of the databases 822 and/or one or more of the LLMs 824 can be included as part of the platform 814 and/or the cloud infrastructure in which the platform 814 is included. While FIG. 8 describes the computing environment 800 as including the LLMs 824, other types of ML models can be included in the computing environment 800, such as an ML model configured for analyzing audio data and/or generating text based on audio data or an ML model configured to generate an execution plan for a group of multiple agent-driven services (or sub-services) included in the platform 814.

Each client device included in the client devices 810 can be any kind of electronic device that is capable of: executing applications; presenting information textually, graphically, and audibly such as via a display and a speaker; collecting information via one or more sensing elements such as image sensors, microphones, tactile sensors, touchscreen displays, and the like; connecting to a communication channel such as the communication channels 812 or a network such as a wireless network, wired network, a public network, a private network, and the like, to send and receive data and information; and/or storing data and information locally in one or more storage mediums of the electronic device and/or in one or more locations that are remote from the electronic device such as a cloud-based storage system, the platform 814, and/or the databases 822. Examples of electronic devices include, and are not limited to, mobile phones, desktop computers, portable computing devices, computers, workstations, laptop computers, tablet computers, and the like.

In some implementations, an application can be installed on, executing on, and/or accessed by a client device included in the client devices 810. The application and/or a user interface of the application can be utilized and/or interacted with (e.g., by an end user) to access, utilize, and/or interact with one or more services provided by the platform 814. The client devices 810 can be configured to receive multiple forms of input such as touch, text, voice, and the like, and the application can be configured to transform that input into one or more messages which can be transmitted or streamed to the platform 814 using one or more communication channels of the communication channels 812. Additionally, the client device can be configured to receive messages, data, and information from the platform 814 using one or more communication channels of the communication channels 812 and the application can be configured to present and/or render the received messages, data, and information in one or more user interfaces of the application. In some cases, the platform 814 receives one or more user queries, such as a user query 805, from the client devices 810. In some cases, the platform 814 provides one or more knowledge-grounded responses, such as knowledge-grounded response data 890, to the client devices 810.

Each communication channel included in the communication channels 812 can be any kind of communication channel that is capable of facilitating communication and the transfer of data and/or information between one or more entities such as the client devices 810, the platform 814, the databases 822, and the LLMs 824 (or other ML models). Examples of communication channels include, and are not limited to, public networks, private networks, the Internet, wireless networks, wired networks, fiber optic networks, local area networks, wide area networks, and the like. The communication channels 812 can be configured to facilitate data and/or information streaming between and among the one or more entities. In some implementations, data and/or information can be streamed using one or more messages and according to one or more protocols. Each of the one or more messages can be a variable length message and each communication channel included in the communication channels 812 can include a stream orchestration layer that can receive the variable length message in accordance with a predefined interface, such as an interface defined using an interface description language like AsyncAPI. Each of the variable length messages can include context information that can be used to determine the route or routes for the variable length message as well as a text or binary payload of arbitrary length. Each of the routes can be configured using a polyglot stream orchestration language that is agnostic to the details of the underlying implementation of the routing tasks and destinations.

Each database included in the databases 822 can be any kind of database that is capable of storing data and/or information and managing data and/or information. Data and/or information stored by each database can include data and/or information generated by, provided by, and/or otherwise obtained by the platform 814. Additionally, or alternatively, data and/or information stored and/or managed by each database can include data and/or information generated by, provided by, and/or otherwise obtained by other sources such as the client devices 810 and/or LLMs 824 (or other ML models). One or more databases that are included in the databases 822 can be part of a platform for storing and managing healthcare information such as electronic health records for patients, electronic records of healthcare providers, and the like, and can store and manage electronic health records for patients of healthcare providers. An example platform is the Oracle Health Millenium Platform. Additionally, one or more databases included in the databases 822 can be provided by, managed by, and/or otherwise included as part of a cloud infrastructure of a cloud service provider (e.g., Oracle Cloud Infrastructure or OCI). Data and/or information stored and/or managed by the databases 822 can be accessed using one or more application programming interfaces (APIs) of the databases 822.

Each LLM included in the LLMs 824 can be any kind of LLM that is capable of obtaining or generating or retrieving one or more results in response to one or more inputs such as one or more machine-learning prompts (hereinafter, “ML prompts” or “prompts”). ML prompts for obtaining or generating or retrieving results from the LLMs 824 can obtained from or generated by or retrieved from or accessed from the client devices 810, the databases 822, the platform 814, and/or one or more other sources such as the Internet. Each ML prompt can be configured to cause the LLMs 824 to perform one or more tasks, which causes one or more results to be provided or generated and the like. ML prompts for the LLMs 824 can be pre-generated (e.g., before they are needed for a particular task) and/or generated in real-time (e.g., without a delay noticeable to a human user). In some implementations, prompts for the LLMs 824 can be engineered to achieve a desired result or results manually and/or by one or more ML models. In some implementations, ML prompts for the LLMs 824 can be engineered on demand (i.e., in real-time and/or as needed) and/or at particular intervals (e.g., once per day, upon log in by authenticated user into the platform 814). Each ML prompt of the one or more ML prompts can include a request, such as a query, for a task to be performed by the LLMs 824. In some cases, an ML prompt can include additional information, such as data generated by one or more services (e.g., agent-driven services) included in the platform 814. The additional information can include information such as one or more ML prompt templates, structured data that is configured to be interpreted (e.g., semantically interpreted) by a computing system component (e.g., an ML model, an agent-driven service, etc.), unstructured data that is configured to be interpreted (e.g., semantically interpreted) by a human, responses from one or more ML models, output data generated by one or more agent-driven services, and/or other information suitable to include in an ML prompt. LLMs included in the LLMs 824 can be pre-trained, fine-tuned, open source, off-the-shelf, licensed, subscribed to, and the like. Additionally, LLMs included in the LLMs 824 can include or have any size context window (e.g., can accept any number of tokens) and can be capable of interpreting complex instructions. One or more LLMs included in the LLMs 824 can be provided by, managed by, and/or otherwise included as part of the platform 814 and/or a cloud infrastructure of a cloud service provider (e.g., Oracle Cloud Infrastructure or OCI) that supports the platform 814. One or more LLMs included in the LLMs 824 can be accessed using one or more APIs of the LLMs 824 and/or a platform hosting or supporting or providing the LLMs 824. In some implementations, one or more additional ML models included in the environment 800 may have one or more characteristics that are similar to characteristics described in regard to the LLMs 824.

The platform 814 can be configured to include various capabilities and provide various services to subscribers (e.g., end users) of the various services. In some implementations, such as in the case of an end user or subscriber being a healthcare provider, the healthcare provider can utilize the various services to facilitate the observation, care, treatment, management, and so on of their patient populations. For example, a healthcare provider can utilize the functionality provided by the various services provided by the platform 814 to examine and/or treat and/or facilitate the examination and/or treatment of a patient; view, edit, and/or manage a patient's electronic health record; perform administrative tasks such as placing medical orders, scheduling appointments, managing patient populations, providing customer service to facilitate operation of a healthcare environment in which the healthcare provider practices, and so on.

In some implementations, the services provided by the platform 814 can include, and are not limited to, a response engine 815 and a knowledge engine 817. In some implementations, one or more services provided by the platform 814, such as the response engine 815 and/or the knowledge engine 817, can be configured to operate as agent-driven services, such as agent-driven services 820. In some implementations, an execution plan guides activities of one or more of the agent-driven services 820 provided by the platform 814. For example, the platform 814 can include, such as included in or in addition to the LLMs 824, a generative AI model (or another suitable ML model included in the environment 800) that is configured to generate (or modify) an execution plan. In this example, the execution plan can describe actions associated with one or more of the agent-driven services 820 provided by the platform 814. Based on the execution plan generated by the example generative AI model, one or more of the agent-driven services 820 can be configured to operate and/or interact with one or more additional ones of the agent-driven services 820. In some implementations, an output of the platform 814 is described by the execution plan. For example, the example generative AI model could be configured to generate an execution plan based on request data associated with the platform 814 (such as request data included in at least one query received by the platform 814 from one or more of the client devices 810). In some cases, the platform 814 and/or the example generative AI model can determine that the request data is associated with at least one agent-driven service of the services 820 provided by the platform 814, such as request data associated with the response engine 815 and/or the knowledge engine 817. In this example, the example generative AI model can generate an execution plan that describes one or more agent tasks for the at least one agent-driven service provided by the platform 814, such as agent tasks for generating a response to a user query and/or generating knowledge-grounded response data. For example, the response engine 815, the knowledge engine 817, and/or additional services in the agent-driven services 820 generates at least one response data object, such as the knowledge-grounded response data 890, based on a combination of multiple data outputs from the response engine 815 and/or the knowledge engine 817. In this example, the knowledge engine 817 generates the knowledge-grounded response data 890 by combining multiple data outputs from the response engine 815 and/or the knowledge engine 817 in a combination that is described by the execution plan.

In some implementations, the generated execution plan can omit instructions for implementing an agent task and include data outlining an agent task (e.g., data outlining one or more data sources, inputs, or requested outputs). In some implementations, one or more service of the agent-driven services 820 can generate its own instructions for implementing an agent task based on the data outlined in the execution plan. For example, an agent-driven service included in (or otherwise associated with) the response engine 815 can construct one or more ML prompts for generating response data, such as by using data outlined in the example execution plan to identify a prompt template (e.g., from a library of templates), a data source (e.g., a data repository storing information related to the user query 805), and one or more of the LLMs 824 (e.g., configured to generate text data summarizing medical information related to the user query 805). As another example, an agent-driven service included in (or otherwise associated with) the knowledge engine 817 can construct one or more ML prompts for generating and/or annotating the knowledge-grounded response data 890, such as by using data outlined in the example execution plan to identify a prompt template, a data source (e.g., data summarized by the response engine 815 and/or the data repository storing information related to the user query 805), and one or more of the LLMs 824 (e.g., configured to determine at least one response annotation using the data summarized by the response engine 815). In some cases, based on the data outlined in the execution plan, the response engine 815 and/or the knowledge engine 817 can be configured to generate respective instructions by which the services 816 and/or 818 can operate and/or interact with one or more additional services provided by the platform 814 (such as, and not limited to, additional services of the agent-driven services 820). Examples of skill-driven and LLM-based and agent-driven digital assistants are described in U.S. Patent Application No. 17,648,376, filed on Jan. 19, 2022, and U.S. patent application Ser. No. 18/624,472, filed on Apr. 2, 2024, each of which are incorporated by reference as if fully set forth herein.

In the platform 814, one or more of the agent-driven services 820 are configured, such as based on one or more generated execution plans, to create and/or annotate one or more portions of the knowledge-grounded response data 890. In the computing environment 800, the agent-driven services 820 can create the knowledge-grounded response data 890 in response to receiving one or more queries, such as a user query 805. To create the knowledge-grounded response data 890, the agent-driven services 820 perform, via the platform 814, one or more of acquiring LLMs, execution plan creation and/or implementation, asset identification (such as identification of one or more model-selected assets 850), and providing the knowledge-grounded response data 890 to one or more additional computing systems, such as to the client device 810. For example, the platform 814 may receive the user query 805 from a particular one of the client device 810. In addition, the platform 814 may generate at least one execution plan based on the user query 805. In some cases, one or more of the response engine 815, the knowledge engine 817, or one or more additional services of the agent-driven services 820 may identify at least one of the LLMs 824 based on the execution plan (or respective portions of the execution plan). In addition, one or more of the response engine 815, the knowledge engine 817, or the one or more additional services of the agent-driven services 820 may identify, such as from the databases 822, at least one asset based on the execution plan (or respective portions of the execution plan). Based on the identified one(s) of the LLMs 824 and/or the identified asset(s), one or more of the response engine 815, the knowledge engine 817, or the one or more additional services of the agent-driven services 820 may generate and/or modify the knowledge-grounded response data 890. In some cases, the knowledge-grounded response data 890 includes a combination of response data and attention cue data, such as response data that responds to a question (or other query type) included in the user query 805 and attention cue data that draws a user's attention to at least a portion of the response data. Examples of response data can include text data, numeric data, image data (e.g., a radiology image), tabulated data (e.g., arranged in a table or other suitable format), or other types of response data suitable for responding to a user query. Examples of attention cue data can include highlighting data (e.g., color text, color background, color-vision deficiency patterns, etc.), font data (e.g., font size, italics, bold, underlining, typeface, etc.), audio data (e.g., automatic speech generation, audible alert data, etc.), haptic data (e.g., vibration, etc.), or other suitable types of attention cue data suitable for drawing user attention to at least a portion of response data.

In some implementations, the response engine 815 can be configured to automatically generate some or all response data that is included in the knowledge-grounded response data 890. For example, by utilizing an execution plan that is generated based on the user query 805, the response engine 815 may identify a first LLM from the LLMs 824 and one or more assets from the databases 822, such as one or more of an asset 850A, an asset 850B, through an asset 850N that are included in the model-selected assets 850. In addition, the response engine 815 may generate one or more ML prompts based on the one or more assets and provide the one or more ML prompts to the first LLM. Based on information received from the first LLM (e.g., in response to the one or more ML prompts), the response engine 815 may generate response data that responds to a question included in the user query 805. For example, if the user query 805 includes a question “How has Ms. Henderson's new blood pressure medication been working?” the response engine 815 may identify, such as from an electronic health record (hereinafter, “EHR”) associated with the patient Ms. Henderson, a group of blood pressure measurements from a time period associated with a blood pressure medication currently prescribed to the patient. The response engine 815 may select the group of blood pressure measurements as information included in the asset 850A. In some cases, the response engine 815 may identify one or more additional assets from the databases 822 and include the additional assets in the model-selected assets 850, such as including in the asset 850B information describing the currently prescribed blood pressure medication or including in the asset 850N information describing additional medical factors for the patient (e.g., an additional diagnosis, a preferred exercise frequency for the patient, etc.) Continuing with this example, the response engine 815 may determine that the first LLM is fine-tuned to summarize information. In addition, the response engine 815 may generate a first ML prompt that includes one or more of the identified assets (e.g., assets 850A through 850N) and provide the first ML prompt to the first LLM. Based on data received from the first LLM, e.g., data summarizing the identified assets included in the first ML prompt, the response engine 815 may generate response data that includes a combination of text and tabulated numeric data, such as a table of blood pressure measurements and a text description of a trend in the blood pressure measurements since the patient Ms. Henderson began taking the currently prescribed blood pressure medication.

In some implementations, the knowledge engine 817 can be configured to automatically generate some or all attention cue data that is included in the knowledge-grounded response data 890. For example, by utilizing the execution plan that is generated based on the user query 805, the knowledge engine 817 may identify a second LLM from the LLMs 824. In addition, the knowledge engine 817 may identify one or more assets, such as one or more the response data generated by the response engine 815 and/or one or more of the assets 850A through 850N. In some cases, the knowledge engine 817 may generate one or more ML prompts based on the one or more assets and provide the one or more ML prompts to the second LLM. Based on information received from the second LLM (e.g., in response to the one or more ML prompts), the knowledge engine 817 may generate attention cue data that draws user attention to at least a portion of the response data generated by the response engine 815. Continuing with the example question “How has Ms. Henderson's new blood pressure medication been working?” the knowledge engine 817 may determine that the second LLM is fine-tuned to identify high-relevance data in one or more assets. In addition, the knowledge engine 817 may generate a second ML prompt that includes one or more of the identified assets (e.g., the response data generated by the response engine 815 and the assets 850A through 850N) and provide the second ML prompt to the second LLM. Based on data received from the second LLM, e.g., data identifying high-relevance data included in the second ML prompt, the knowledge engine 817 may generate attention cue data that draws attention to the high-relevance data, such as color highlighting that draws attention to a trend in the blood pressure measurements and a bold font style that draws attention to information describing a possible interaction of the currently prescribed blood pressure medication with an additional medication frequently prescribed for an additional diagnosis of the patient. In addition, the knowledge engine 817 may generate or modify the knowledge-grounded response data 890 to include the attention cue data, e.g., modifying the knowledge-grounded response data 890 to apply the color highlighting to at least a portion of the tabulated numeric data and the bold font style to at least a portion of the text data. In some cases, the knowledge engine 817 may modify the knowledge-grounded response data 890 to include additional response data, such as interactive reference data (e.g., a URL address) that provides one or more references describing a source of information included in the knowledge-grounded response data 890. Continuing with the above example, the knowledge engine 817 may generate first interactive reference data that provides a first reference to one or more EHRs including blood pressure measurements for the patient. In addition, the knowledge engine 817 may generate second interactive reference data that provides a second reference to medication information (e.g., a medication reference database) describing possible interactions of the currently prescribed blood pressure medication.

In some implementations, the platform 814 (or a component thereof) may provide the knowledge-grounded response data 890 to one or more additional computing systems. For example, the platform 814 may identify a particular client device of the client devices 810 from which the user query 805 was received. In addition, the platform 814 may provide the knowledge-grounded response data 890 to the particular client device. In some cases, the particular client device is configured to perform one or more operations based on the knowledge-grounded response data 890, such as operations related to displaying the combination of the response data and the attention cue data included in the knowledge-grounded response data 890. For example, the particular client device may be configured to display the response data as annotated by the attention cue data, e.g., the table of blood pressure measurements and the text description as annotated by the color highlighting, the bold font style, and the interactive reference data. In addition, the particular client device may be configured to receive additional input data based on the knowledge-grounded response data 890, such as a user input indicating a selection of at least a portion of the knowledge-grounded response data 890. For example, responsive to receiving a user selection input of the first interactive reference data, the particular client device may be configured to send, to the platform 814, a request to receive at least a portion of the first reference, such as the one or more EHRs (or a portion thereof) including blood pressure measurements for the patient. In addition, responsive to receiving an additional user selection input of the second interactive reference data, the particular client device may be configured to send, to the platform 814, an additional request to receive at least a portion of the second reference, such as the medication information (or a portion thereof) describing possible interactions of the currently prescribed blood pressure medication. In some cases, the data architecture and/or configuration of the platform 814, such as the combination of the response engine 815 and the knowledge engine 817 and/or combination of some or all described features thereof, can improve user comprehension of information provided in response to user queries, such as the user query 805. For example, the combination of the response data with the attention cue data, such as included in the knowledge-grounded response data 890, can improve comprehension or reduce reading time by a user, such as by drawing the user's attention to portions of the response data that are annotated by the attention cue data. In addition, the combination of the response data with the interactive reference data that is included in the attention cue data, such as included in the knowledge-grounded response data 890, can improve user trust in the response data by facilitating fast identification of potentially inaccurate data (e.g., hallucinations) generated by one or more of the LLMs 824, such as by providing fast access to reference information via the interactive reference data.

In FIG. 8, the response engine 815 and the knowledge engine 817 are described as utilizing a particular execution plan (e.g., respective portions of a same execution plan), and other implementations are possible. For example, a cloud service provider platform may generate a respective execution plan for each particular agent-driven service that is included in (or otherwise utilized by) the example cloud service provider platform. In FIG. 8, the response engine 815 and the knowledge engine 817 are described as respectively identifying the first LLM and the second LLM from the LLMs 824, and other implementations are possible. For example, in various instances, the response engine 815 and the knowledge engine 817 (or others of the agent-driven services 820) may identify from the LLMs 824 at least one same LLM, at least one different LLM, and/or a combination of different and same LLMs.

In some instances, the agent-driven services 820 can be utilized to access pre-trained and/or fine-tuned ML models, such as one or more of the LLMs 824. The pre-trained ML models serve as foundational elements, possessing extensive language understanding derived from vast datasets. This capability enables the models to generate coherent responses across various topics, facilitating transfer learning. Pre-trained models offer cost-effectiveness and flexibility, which allows for scalable improvements and continuous pre-training with new data, often establishing benchmarks in natural language processing tasks. Conversely, fine-tuned models are specifically trained for tasks or industries (e.g., plan creation utilizing the LLM's in-context learning capability, knowledge or information retrieval on behalf of an agent, response generation for human-like conversation, etc.), enhancing their performance on specific applications and enabling efficient learning from smaller, specialized datasets. Fine-tuning provides advantages such as task specialization, data efficiency, quicker training times, model customization, and resource efficiency. In some embodiments, fine-tuning may be particularly advantageous for niche applications and ongoing enhancement. In other instances, the agent-driven services 820 can be utilized to pre-train and/or fine-tune the LLMs. The agent-driven services 820, or any subset thereof, may be standalone or part of a machine-learning operationalization framework, inclusive of hardware components like processors (e.g., CPU, GPU, TPU, FPGA, or any combination), memory, and storage. This framework operates software or computer program instructions (e.g., TensorFlow, PyTorch, Keras, etc.) to execute arithmetic, logic, input/output commands for training, validating, and deploying machine-learning models in a production environment. In certain instances, the agent-driven services 820 implement the training, validating, and deploying of the models using a cloud platform such as Oracle Cloud Infrastructure (OCI). In some cases, leveraging a cloud platform can make machine-learning more accessible, flexible, and cost-effective, which can facilitate faster model development and deployment for developers.

Although not shown, the platform 814 can include other capabilities and services such as authentication services, management services, task management services, notification services, and the like. The various capabilities and services of the platform 814 can be implemented utilizing one or more computing resources and/or servers of the platform 814 and provided by the platform 814 by way of subscriptions. Additionally, or alternatively, while FIG. 8 shows the services of the platform 814 as being separate services, one or more of the services can be combined with other services and/or be considered to be a sub-service of another service. In some implementations, a particular service in the agent-driven services 820 may utilize an output from another service in the agent-driven services 820, such as to facilitate quick completion of one or more operations by the particular service. For example, as shown in FIG. 8, one or more of the response engine 815 or the knowledge engine 817 can include at least one sub-service. In addition, one or more of the response engine 815 or the knowledge engine 817 can access one or more outputs from one or more services of the agent-driven services 820, such as agent output data 830. In some cases, the availability of the agent output data 830 to multiple services in the agent-driven services 820, such as at least the response engine 815 and the knowledge engine 817, can improve response time by the multiple services in the agent-driven services 820. For example, the response engine 815 and the knowledge engine 817 may provide one or more outputs with decreased response time and decreased use of computing resources (e.g., processing resources, memory resources, ML model resources, etc.) by using some or all of the agent output data 830 as input data.

FIG. 8 depicts the structured data 832 and the unstructured data 834 as being included in the agent output data 830, and other implementations are possible, such as one or more of the data 832 or 834 being included in the model-selected assets 850. In some cases, one or more of the structured data 832 and the unstructured data 834 is an output from one or more additional services of the agent-driven services 820, such as additional services configured for generating text data based on audio data (e.g., transcription of spoken conversation between a patient and a healthcare provider), identifying EHRs for a particular patient, or other tasks suitable to be performed by the agent-driven services 820. In FIG. 8, the structured data 832 can include data that is structured to be interpreted by a computing device, such as database records, JavaScript data objects, or other data objects (e.g., included in one or more EHRs) that are intended for computer interpretation (e.g., not intended for human interpretation). In FIG. 8, the unstructured data 834 can include data that lacks a structure interpreted by a computing device, such as patient appointment notes (e.g., Subjective, Objective, Assessment, and Plan notes, “SOAP notes”), medical reference materials (e.g., medication documentation, surgical procedure guidelines, etc.), or other types of information that are intended for human interpretation.

In the computing environment 800, the response engine 815 can include at least one sub-service, such as a response data prioritization service 816. In some cases, the response data prioritization service 816 may access at least one of the agent output data 830, such as one or more of the structured data 832 or the unstructured data 834. In addition, the response data prioritization service 816 may evaluate portions of the structured data 832 or the unstructured data 834 for potential inclusion in response data. For example, the response data prioritization service 816 may evaluate a data portion to determine whether the data is relevant to a question or other information in the user query 805. In some cases, the response data prioritization service 816 may send one or more portions of the structured data 832 or the unstructured data 834 to at least one LLM of the LLMs 824, such as in an ML prompt. In some implementations, the ML prompt also includes data corresponding to the user query 805, such as a question included in the user query 805 or additional data (e.g., determined by a service in the agent-driven services 820), such as additional data corresponding to the user query 805.

In the computing environment 800, the response data prioritization service 816 may receive, from the at least one LLM, multiple data portions that are extracted from one or more of the structured data 832 or the unstructured data 834. In addition, the response data prioritization service 816 may determine a relevance status for each of the multiple data portions, such as by comparing each data portion to one or more relevance threshold values. In some cases, the response data prioritization service 816 may determine, such as by determining a similarity (e.g., semantic similarity) between each data portion and query data (e.g., information associated with the user query 805), whether each data portion exceeds (or fulfill another relationship with) the one or more relevance threshold values. For example, based on a comparison to a first relevance threshold value, the response data prioritization service 816 may identify various data portions as high-relevance data, such as high-relevance data that directly answers one or more questions included in the user query 805. In some cases, the response data prioritization service 816 may select one or more of the LLMs 824 to modify some of the high-relevance data before inclusion in response data 836. For example, the response data prioritization service 816 may provide a first portion of the high-relevance data to a first LLM (e.g., from the LLMs 824) that is fine-tuned to summarize received information in text summary data. Examples of text summary data can include paragraphs, single sentences, or other human-readable text data that summarizes larger amounts of information. In addition, the response data prioritization service 816 may modify the response data 836 to include the text summary data which summarizes the high-relevance data. As another example, the response data prioritization service 816 may provide a second portion of the high-relevance data to a second LLM (e.g., from the LLMs 824) that is fine-tuned to arrange received information as tabulated data, such as multiple items of information that are intended to be interpreted as a group. Examples of tabulated data can include tables, bulleted lists, numbered lists, or other organized arrangements of multiple items of information intended to be interpreted as a group. Examples of information that could be arranged as tabulated data can include a group of lab results, a group of comparison medications (e.g., generics, non-generics, etc.), a group of potential side effects of a surgical procedure, or other groups of information items. In addition, the response data prioritization service 816 may modify the response data 836 to include the tabulated data in which the high-relevance data is arranged.

In some implementations, based on a comparison to a second relevance threshold value, the response data prioritization service 816 may identify one or more data portions (e.g., extracted from one or more of the structured data 832 or the unstructured data 834) as medium-relevance data. In some cases, the medium-relevance data can include supplemental data that does not directly answer one or more questions included in the user query 805 and which provides additional data about a topic identified in the one or more questions. Examples of supplemental data can include information about a diagnosed condition, information about a patient circumstance (e.g., a high-exercise lifestyle, a preference to avoid injected medications, etc.), or other types of information that are generally related to a question. In some cases, the response data prioritization service 816 may select one or more of the LLMs 824 to modify some of the medium-relevance data before inclusion in the response data 836. For example, the response data prioritization service 816 may provide a portion of the medium-relevance data to the first LLM that is fine-tuned to summarize received information in text summary data. In addition, the response data prioritization service 816 may modify the response data 936 to include additional text summary data which summarizes the medium-relevance data.

In the computing environment 800, the knowledge engine 817 can include at least one sub-service, such as one or more of an annotation selection service 818 or a display preparation service 819. In some cases, one or more of the annotation selection service 818 or the display preparation service 819 may access at least one of the agent output data 830, such as the response data 836 that is generated by the response engine 815. Examples of computer-implemented instructions related to display preparation service can include hypertext markup language (HTML) instructions, extensible markup language (XML) instructions, or other suitable types of instructions for implementing data display (e.g., visual display, audio display, etc.) via one or more user interface devices.

In the computing environment 800, the annotation selection service 818 may provide one or more portions of the response data 836 to at least one LLM of the LLMs 824, such as in an ML prompt. In some implementations, the ML prompt also includes additional data (e.g., determined by a service in the agent-driven services 820), such as additional data corresponding to one or more of the user query 805, the structured data 832, or the unstructured data 834. For example, the at least one LLM may be fine-tuned to identify, in the response data 836, one or more portions of data that have a relatively high similarity to data included in one or more of the user query 805, the structured data 832, or the unstructured data 834, such as a portion of high-relevance text summary data in the response data 836 that has a high similarity to text data of a question included in the user query 805. In some cases, the annotation selection service 818 may receive, from the at least one LLM, data identifying at least one portion of the response data 836 for annotation. In addition, the annotation selection service 818 may determine one or more types of annotations to apply to the identified portion of the response data 836, such as annotations for highlighting, font styles, or other types of annotations that can be applied to response data. In some implementations, the annotation selection service 818 may determine at least one type of annotation that applies interactive reference data to the identified portion of the response data 836. For example, the annotation selection service 818 may determine one or more sources for the identified portion of the response data 836, such as a source document and/or source database associated with one or more of the structured data 832 or the unstructured data 834. Based on the determined one or more sources, the annotation selection service 818 may generate interactive reference data that indicates the source(s) for the identified portion of the response data 836. In some cases, the annotation selection service 818 may identify source address data associated with the source(s) for the identified portion of the response data 836. Examples of source address data can include a network address (e.g., a URL, a MAC address), computing component identification data (e.g., identification of a particular database, etc.), document identification data (e.g., identification of a particular document, identification of a section within a document, etc.), or other types of address data that can identify a location (or other identification type) for a source repository.

In the computing environment 800, the display preparation service 819 may identify one or more associated portions of response data. In addition, the display preparation service 819 may generate one or more computer-implemented instructions that combine the associated portions of response data for presentation via one or more user interface devices. In addition, the display preparation service 819 may generate at least one computer-implemented instruction that combines the associated portions, such as an HTML instruction (or other suitable instruction type) that applies a bold typeface to the sentence. As another example, the display preparation service 819 may determine an additional association between a second portion of the response data 836 that indicates tabulated data, such as a set of blood pressure measurements, and an additional annotation including interactive reference data, such as a patient chart that is a source document for the set of blood pressure measurements. In addition, the display preparation service 819 may generate at least one additional computer-implemented instruction that combines the additional associated portions, such as an additional HTML instruction (or other suitable instruction type) that applies an interactive link to the tabulated set of blood pressure measurements, e.g., the interactive link is directed to the patient chart. The computing environment 800 depicted in FIG. 8 is merely exemplary and not intended to unduly limit the scope of claimed embodiments. One of ordinary skill in the art would recognize many possible variations, alternatives, and modifications. For example, in some implementations, the computing environment 800 900 can be implemented using more or fewer services than those shown in FIGS. 8, may combine two or more services, or may have a different configuration or arrangement of services.

### Examples of Cloud Infrastructure Architectures

The term cloud service is generally used to refer to a service that is made available by a cloud service provider (CSP) to users (e.g., cloud service customers) on demand (e.g., via a subscription model) using systems and infrastructure (cloud infrastructure) provided by the CSP. Typically, the servers and systems that make up the CSP's infrastructure are separate from the user's own on-premise servers and systems. Users can thus avail themselves of cloud services provided by the CSP without having to purchase separate hardware and software resources for the services. Cloud services are designed to provide a subscribing user easy, scalable access to applications and computing resources without the user having to invest in procuring the infrastructure that is used for providing the services.

There are several cloud service providers that offer various types of cloud services. As discussed herein, there are various types or models of cloud services including IaaS, software as a service (SaaS), platform as a service (PaaS), and others. A user can subscribe to one or more cloud services provided by a CSP. The user can be any entity such as an individual, an organization, an enterprise, and the like. When a user subscribes to or registers for a service provided by a CSP, a tenancy or an account is created for that user. The user can then, via this account, access the subscribed-to one or more cloud resources associated with the account.

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

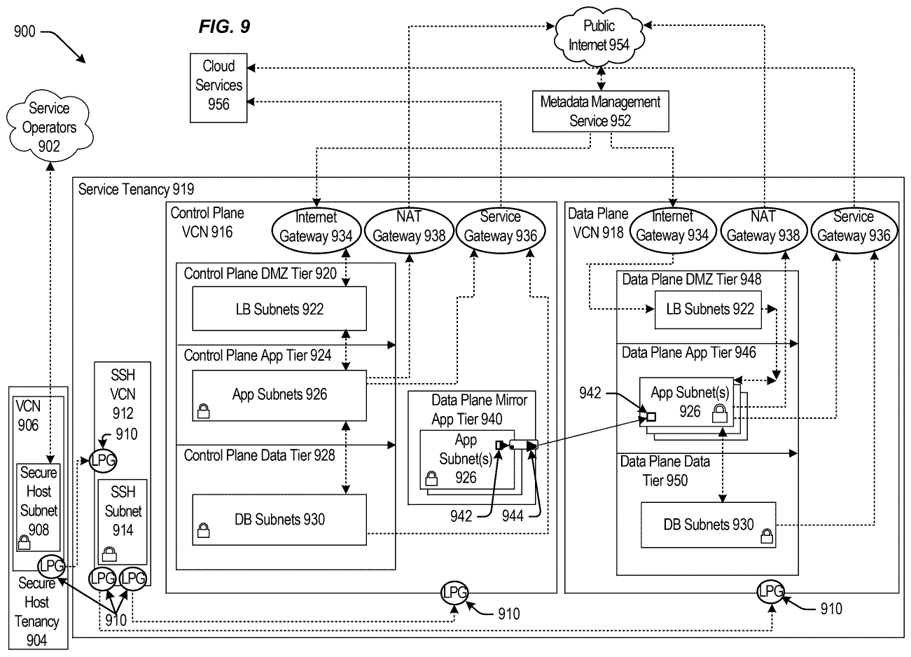

FIG. 9 is a block diagram 900 illustrating an example pattern of an IaaS architecture, according to at least one embodiment. Service operators 902 can be communicatively coupled to a secure host tenancy 904 that can include a virtual cloud network (VCN) 906 and a secure host subnet 908. In some examples, the service operators 902 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 906 and/or the Internet.

The VCN 906 can include a local peering gateway (LPG) 910 that can be communicatively coupled to a secure shell (SSH) VCN 912 via an LPG 910 contained in the SSH VCN 912. The SSH VCN 912 can include an SSH subnet 914, and the SSH VCN 912 can be communicatively coupled to a control plane VCN 916 via the LPG 910 contained in the control plane VCN 916. Also, the SSH VCN 912 can be communicatively coupled to a data plane VCN 918 via an LPG 910. The control plane VCN 916 and the data plane VCN 918 can be contained in a service tenancy 919 that can be owned and/or operated by the IaaS provider.

The control plane VCN 916 can include a control plane demilitarized zone (DMZ) tier 920 that acts as a perimeter network (e.g., portions of a corporate network between the corporate intranet and external networks). The DMZ-based servers may have restricted responsibilities and help keep breaches contained. Additionally, the DMZ tier 920 can include one or more load balancer (LB) subnet(s) 922, a control plane app tier 924 that can include app subnet(s) 926, a control plane data tier 928 that can include database (DB) subnet(s) 930 (e.g., frontend DB subnet(s) and/or backend DB subnet(s)). The LB subnet(s) 922 contained in the control plane DMZ tier 920 can be communicatively coupled to the app subnet(s) 926 contained in the control plane app tier 924 and an Internet gateway 934 that can be contained in the control plane VCN 916, and the app subnet(s) 926 can be communicatively coupled to the DB subnet(s) 930 contained in the control plane data tier 928 and a service gateway 936 and a network address translation (NAT) gateway 938. The control plane VCN 916 can include the service gateway 936 and the NAT gateway 938.

The control plane VCN 916 can include a data plane mirror app tier 940 that can include app subnet(s) 926. The app subnet(s) 926 contained in the data plane mirror app tier 940 can include a virtual network interface controller (VNIC) 942 that can execute a compute instance 944. The compute instance 944 can communicatively couple the app subnet(s) 926 of the data plane mirror app tier 940 to app subnet(s) 926 that can be contained in a data plane app tier 946.

The data plane VCN 918 can include the data plane app tier 946, a data plane DMZ tier 948, and a data plane data tier 950. The data plane DMZ tier 948 can include LB subnet(s) 922 that can be communicatively coupled to the app subnet(s) 926 of the data plane app tier 946 and the Internet gateway 934 of the data plane VCN 918. The app subnet(s) 926 can be communicatively coupled to the service gateway 936 of the data plane VCN 918 and the NAT gateway 938 of the data plane VCN 918. The data plane data tier 950 can also include the DB subnet(s) 930 that can be communicatively coupled to the app subnet(s) 926 of the data plane app tier 946.

The Internet gateway 934 of the control plane VCN 916 and of the data plane VCN 918 can be communicatively coupled to a metadata management service 952 that can be communicatively coupled to public Internet 954. Public Internet 954 can be communicatively coupled to the NAT gateway 938 of the control plane VCN 916 and of the data plane VCN 918. The service gateway 936 of the control plane VCN 916 and of the data plane VCN 918 can be communicatively coupled to cloud services 956.

In some examples, the service gateway 936 of the control plane VCN 916 or of the data plane VCN 918 can make application programming interface (API) calls to cloud services 956 without going through public Internet 954. The API calls to cloud services 956 from the service gateway 936 can be one-way: the service gateway 936 can make API calls to cloud services 956, and cloud services 956 can send requested data to the service gateway 936. But, cloud services 956 may not initiate API calls to the service gateway 936.

In some examples, the secure host tenancy 904 can be directly connected to the service tenancy 919, which may be otherwise isolated. The secure host subnet 908 can communicate with the SSH subnet 914 through an LPG 910 that may enable two-way communication over an otherwise isolated system. Connecting the secure host subnet 908 to the SSH subnet 914 may give the secure host subnet 908 access to other entities within the service tenancy 919.

The control plane VCN 916 may allow users of the service tenancy 919 to set up or otherwise provision desired resources. Desired resources provisioned in the control plane VCN 916 may be deployed or otherwise used in the data plane VCN 918. In some examples, the control plane VCN 916 can be isolated from the data plane VCN 918, and the data plane mirror app tier 940 of the control plane VCN 916 can communicate with the data plane app tier 946 of the data plane VCN 918 via VNICs 942 that can be contained in the data plane mirror app tier 940 and the data plane app tier 946.

In some examples, users of the system, or customers, can make requests, for example create, read, update, or delete (CRUD) operations, through public Internet 954 that can communicate the requests to the metadata management service 952. The metadata management service 952 can communicate the request to the control plane VCN 916 through the Internet gateway 934. The request can be received by the LB subnet(s) 922 contained in the control plane DMZ tier 920. The LB subnet(s) 922 may determine that the request is valid, and in response to this determination, the LB subnet(s) 922 can transmit the request to app subnet(s) 926 contained in the control plane app tier 924. If the request is validated and requires a call to public Internet 954, the call to public Internet 954 may be transmitted to the NAT gateway 938 that can make the call to public Internet 954. Metadata that may be desired to be stored by the request can be stored in the DB subnet(s) 930.

In some examples, the data plane mirror app tier 940 can facilitate direct communication between the control plane VCN 916 and the data plane VCN 918. For example, changes, updates, or other suitable modifications to configuration may be desired to be applied to the resources contained in the data plane VCN 918. Via a VNIC 942, the control plane VCN 916 can directly communicate with, and can thereby execute the changes, updates, or other suitable modifications to configuration to, resources contained in the data plane VCN 918.

In some embodiments, the control plane VCN 916 and the data plane VCN 918 can be contained in the service tenancy 919. In this case, the user, or the customer, of the system may not own or operate either the control plane VCN 916 or the data plane VCN 918. Instead, the IaaS provider may own or operate the control plane VCN 916 and the data plane VCN 918, both of which may be contained in the service tenancy 919. This embodiment can enable isolation of networks that may prevent users or customers from interacting with other users', or other customers', resources. Also, this embodiment may allow users or customers of the system to store databases privately without needing to rely on public Internet 954, which may not have a desired level of threat prevention, for storage.

In other embodiments, the LB subnet(s) 922 contained in the control plane VCN 916 can be configured to receive a signal from the service gateway 936. In this embodiment, the control plane VCN 916 and the data plane VCN 918 may be configured to be called by a customer of the IaaS provider without calling public Internet 954. Customers of the IaaS provider may desire this embodiment since database(s) that the customers use may be controlled by the IaaS provider and may be stored on the service tenancy 919, which may be isolated from public Internet 954.

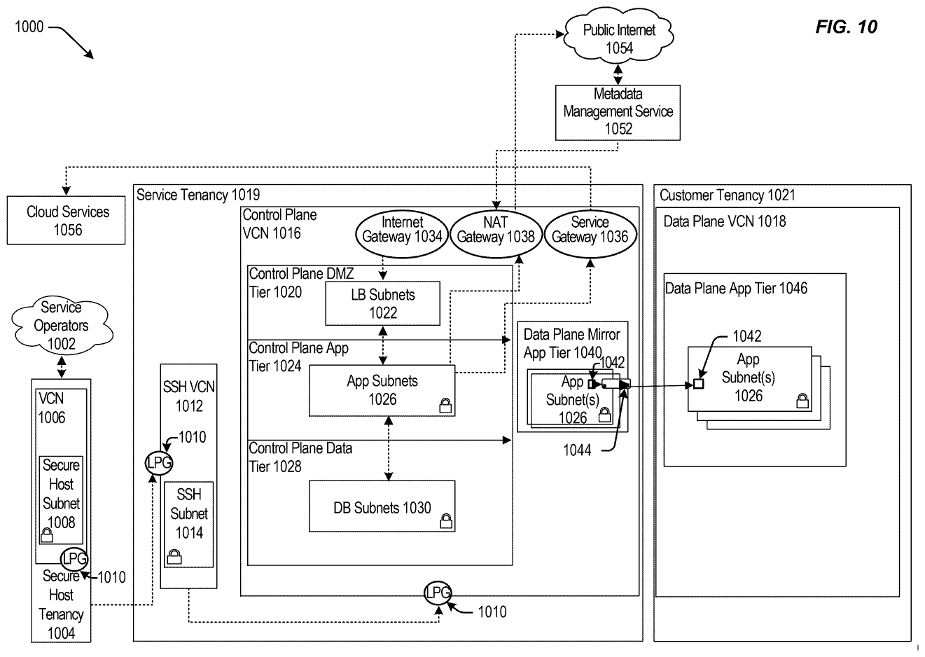

FIG. 10 is a block diagram 1000 illustrating another example pattern of an IaaS architecture, according to at least one embodiment. Service operators 1002 (e.g., service operators 902 of FIG. 9) can be communicatively coupled to a secure host tenancy 1004 (e.g., the secure host tenancy 904 of FIG. 9) that can include a virtual cloud network (VCN) 1006 (e.g., the VCN 906 of FIG. 9) and a secure host subnet 1008 (e.g., the secure host subnet 908 of FIG. 9). The VCN 1006 can include a local peering gateway (LPG) 1010 (e.g., the LPG 910 of FIG. 9) that can be communicatively coupled to a secure shell (SSH) VCN 1012 (e.g., the SSH VCN 912 of FIG. 9) via an LPG 910 contained in the SSH VCN 1012. The SSH VCN 1012 can include an SSH subnet 1014 (e.g., the SSH subnet 914 of FIG. 9), and the SSH VCN 1012 can be communicatively coupled to a control plane VCN 1016 (e.g., the control plane VCN 916 of FIG. 9) via an LPG 1010 contained in the control plane VCN 1016. The control plane VCN 1016 can be contained in a service tenancy 1019 (e.g., the service tenancy 919 of FIG. 9), and the data plane VCN 1018 (e.g., the data plane VCN 918 of FIG. 9) can be contained in a customer tenancy 1021 that may be owned or operated by users, or customers, of the system.

The control plane VCN 1016 can include a control plane DMZ tier 1020 (e.g., the control plane DMZ tier 920 of FIG. 9) that can include LB subnet(s) 1022 (e.g., LB subnet(s) 922 of FIG. 9), a control plane app tier 1024 (e.g., the control plane app tier 924 of FIG. 9) that can include app subnet(s) 1026 (e.g., app subnet(s) 926 of FIG. 9), a control plane data tier 1028 (e.g., the control plane data tier 928 of FIG. 9) that can include database (DB) subnet(s) 1030 (e.g., similar to DB subnet(s) 930 of FIG. 9). The LB subnet(s) 1022 contained in the control plane DMZ tier 1020 can be communicatively coupled to the app subnet(s) 1026 contained in the control plane app tier 1024 and an Internet gateway 1034 (e.g., the Internet gateway 934 of FIG. 9) that can be contained in the control plane VCN 1016, and the app subnet(s) 1026 can be communicatively coupled to the DB subnet(s) 1030 contained in the control plane data tier 1028 and a service gateway 1036 (e.g., the service gateway 936 of FIG. 9) and a network address translation (NAT) gateway 1038 (e.g., the NAT gateway 938 of FIG. 9). The control plane VCN 1016 can include the service gateway 1036 and the NAT gateway 1038.

The control plane VCN 1016 can include a data plane mirror app tier 1040 (e.g., the data plane mirror app tier 940 of FIG. 9) that can include app subnet(s) 1026. The app subnet(s) 1026 contained in the data plane mirror app tier 1040 can include a virtual network interface controller (VNIC) 1042 (e.g., the VNIC of 942) that can execute a compute instance 1044 (e.g., similar to the compute instance 944 of FIG. 9). The compute instance 1044 can facilitate communication between the app subnet(s) 1026 of the data plane mirror app tier 1040 and the app subnet(s) 1026 that can be contained in a data plane app tier 1046 (e.g., the data plane app tier 946 of FIG. 9) via the VNIC 1042 contained in the data plane mirror app tier 1040 and the VNIC 1042 contained in the data plane app tier 1046.

The Internet gateway 1034 contained in the control plane VCN 1016 can be communicatively coupled to a metadata management service 1052 (e.g., the metadata management service 952 of FIG. 9) that can be communicatively coupled to public Internet 1054 (e.g., public Internet 954 of FIG. 9). Public Internet 1054 can be communicatively coupled to the NAT gateway 1038 contained in the control plane VCN 1016. The service gateway 1036 contained in the control plane VCN 1016 can be communicatively coupled to cloud services 1056 (e.g., cloud services 956 of FIG. 9).

In some examples, the data plane VCN 1018 can be contained in the customer tenancy 1021. In this case, the IaaS provider may provide the control plane VCN 1016 for each customer, and the IaaS provider may, for each customer, set up a unique compute instance 1044 that is contained in the service tenancy 1019. Each compute instance 1044 may allow communication between the control plane VCN 1016, contained in the service tenancy 1019, and the data plane VCN 1018 that is contained in the customer tenancy 1021. The compute instance 1044 may allow resources, that are provisioned in the control plane VCN 1016 that is contained in the service tenancy 1019, to be deployed or otherwise used in the data plane VCN 1018 that is contained in the customer tenancy 1021.

In other examples, the customer of the IaaS provider may have databases that live in the customer tenancy 1021. In this example, the control plane VCN 1016 can include the data plane mirror app tier 1040 that can include app subnet(s) 1026. The data plane mirror app tier 1040 can reside in the data plane VCN 1018, but the data plane mirror app tier 1040 may not live in the data plane VCN 1018. That is, the data plane mirror app tier 1040 may have access to the customer tenancy 1021, but the data plane mirror app tier 1040 may not exist in the data plane VCN 1018 or be owned or operated by the customer of the IaaS provider. The data plane mirror app tier 1040 may be configured to make calls to the data plane VCN 1018 but may not be configured to make calls to any entity contained in the control plane VCN 1016. The customer may desire to deploy or otherwise use resources in the data plane VCN 1018 that are provisioned in the control plane VCN 1016, and the data plane mirror app tier 1040 can facilitate the desired deployment, or other usage of resources, of the customer.

In some embodiments, the customer of the IaaS provider can apply filters to the data plane VCN 1018. In this embodiment, the customer can determine what the data plane VCN 1018 can access, and the customer may restrict access to public Internet 1054 from the data plane VCN 1018. The IaaS provider may not be able to apply filters or otherwise control access of the data plane VCN 1018 to any outside networks or databases. Applying filters and controls by the customer onto the data plane VCN 1018, contained in the customer tenancy 1021, can help isolate the data plane VCN 1018 from other customers and from public Internet 1054.

In some embodiments, cloud services 1056 can be called by the service gateway 1036 to access services that may not exist on public Internet 1054, on the control plane VCN 1016, or on the data plane VCN 1018. The connection between cloud services 1056 and the control plane VCN 1016 or the data plane VCN 1018 may not be live or continuous. Cloud services 1056 may exist on a different network owned or operated by the IaaS provider. Cloud services 1056 may be configured to receive calls from the service gateway 1036 and may be configured to not receive calls from public Internet 1054. Some cloud services 1056 may be isolated from other cloud services 1056, and the control plane VCN 1016 may be isolated from cloud services 1056 that may not be in the same region as the control plane VCN 1016. For example, the control plane VCN 1016 may be located in “Region 1,” and cloud service “Deployment 9,” may be located in Region 1 and in “Region 2.” If a call to Deployment 9 is made by the service gateway 1036 contained in the control plane VCN 1016 located in Region 1, the call may be transmitted to Deployment 9 in Region 1. In this example, the control plane VCN 1016, or Deployment 9 in Region 1, may not be communicatively coupled to, or otherwise in communication with, Deployment 9 in Region 2.

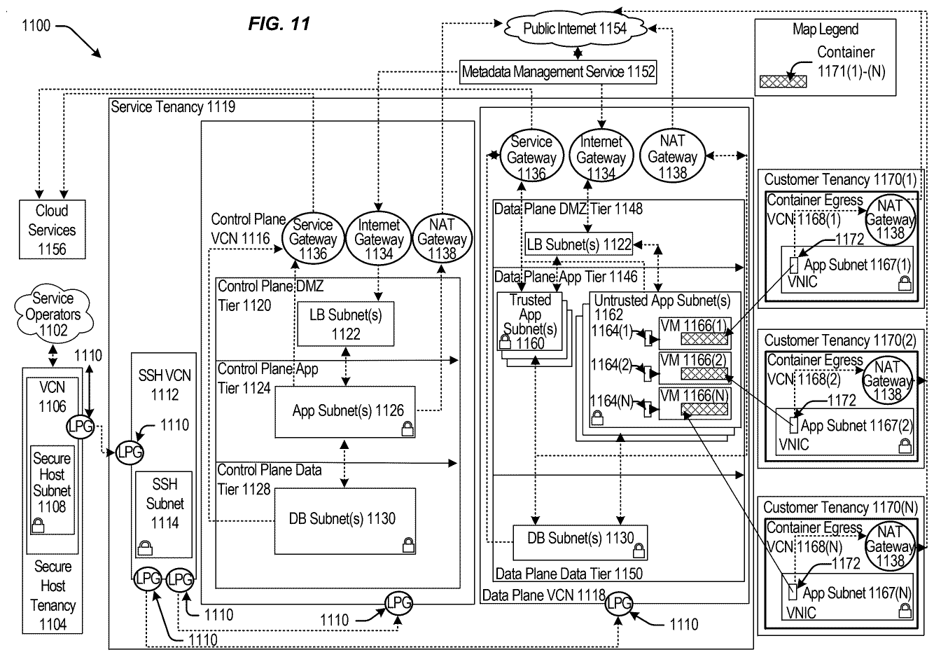

FIG. 11 is a block diagram 1100 illustrating another example pattern of an IaaS architecture, according to at least one embodiment. Service operators 1102 (e.g., service operators 902 of FIG. 9) can be communicatively coupled to a secure host tenancy 1104 (e.g., the secure host tenancy 904 of FIG. 9) that can include a virtual cloud network (VCN) 1106 (e.g., the VCN 906 of FIG. 9) and a secure host subnet 1108 (e.g., the secure host subnet 908 of FIG. 9). The VCN 1106 can include an LPG 1110 (e.g., the LPG 910 of FIG. 9) that can be communicatively coupled to an SSH VCN 1112 (e.g., the SSH VCN 912 of FIG. 9) via an LPG 1110 contained in the SSH VCN 1112. The SSH VCN 1112 can include an SSH subnet 1114 (e.g., the SSH subnet 914 of FIG. 9), and the SSH VCN 1112 can be communicatively coupled to a control plane VCN 1116 (e.g., the control plane VCN 916 of FIG. 9) via an LPG 1110 contained in the control plane VCN 1116 and to a data plane VCN 1118 (e.g., the data plane 918 of FIG. 9) via an LPG 1110 contained in the data plane VCN 1118. The control plane VCN 1116 and the data plane VCN 1118 can be contained in a service tenancy 1119 (e.g., the service tenancy 919 of FIG. 9).

The control plane VCN 1116 can include a control plane DMZ tier 1120 (e.g., the control plane DMZ tier 920 of FIG. 9) that can include load balancer (LB) subnet(s) 1122 (e.g., LB subnet(s) 922 of FIG. 9), a control plane app tier 1124 (e.g., the control plane app tier 924 of FIG. 9) that can include app subnet(s) 1126 (e.g., similar to app subnet(s) 926 of FIG. 9), a control plane data tier 1128 (e.g., the control plane data tier 928 of FIG. 9) that can include DB subnet(s) 1130. The LB subnet(s) 1122 contained in the control plane DMZ tier 1120 can be communicatively coupled to the app subnet(s) 1126 contained in the control plane app tier 1124 and to an Internet gateway 1134 (e.g., the Internet gateway 934 of FIG. 9) that can be contained in the control plane VCN 1116, and the app subnet(s) 1126 can be communicatively coupled to the DB subnet(s) 1130 contained in the control plane data tier 1128 and to a service gateway 1136 (e.g., the service gateway of FIG. 9) and a network address translation (NAT) gateway 1138 (e.g., the NAT gateway 938 of FIG. 9). The control plane VCN 1116 can include the service gateway 1136 and the NAT gateway 1138.

The data plane VCN 1118 can include a data plane app tier 1146 (e.g., the data plane app tier 946 of FIG. 9), a data plane DMZ tier 1148 (e.g., the data plane DMZ tier 948 of FIG. 9), and a data plane data tier 1150 (e.g., the data plane data tier 950 of FIG. 9). The data plane DMZ tier 1148 can include LB subnet(s) 1122 that can be communicatively coupled to trusted app subnet(s) 1160 and untrusted app subnet(s) 1162 of the data plane app tier 1146 and the Internet gateway 1134 contained in the data plane VCN 1118. The trusted app subnet(s) 1160 can be communicatively coupled to the service gateway 1136 contained in the data plane VCN 1118, the NAT gateway 1138 contained in the data plane VCN 1118, and DB subnet(s) 1130 contained in the data plane data tier 1150. The untrusted app subnet(s) 1162 can be communicatively coupled to the service gateway 1136 contained in the data plane VCN 1118 and DB subnet(s) 1130 contained in the data plane data tier 1150. The data plane data tier 1150 can include DB subnet(s) 1130 that can be communicatively coupled to the service gateway 1136 contained in the data plane VCN 1118.

The untrusted app subnet(s) 1162 can include one or more primary VNICs 1164(1)-(N) that can be communicatively coupled to tenant virtual machines (VMs) 1166(1)-(N). Each tenant VM 1166(1)-(N) can be communicatively coupled to a respective app subnet 1167(1)-(N) that can be contained in respective container egress VCNs 1168(1)-(N) that can be contained in respective customer tenancies 1170(1)-(N). Respective secondary VNICs 1172(1)-(N) can facilitate communication between the untrusted app subnet(s) 1162 contained in the data plane VCN 1118 and the app subnet contained in the container egress VCNs 1168(1)-(N). Each container egress VCNs 1168(1)-(N) can include a NAT gateway 1138 that can be communicatively coupled to public Internet 1154 (e.g., public Internet 954 of FIG. 9).

The Internet gateway 1134 contained in the control plane VCN 1116 and contained in the data plane VCN 1118 can be communicatively coupled to a metadata management service 1152 (e.g., the metadata management system 952 of FIG. 9) that can be communicatively coupled to public Internet 1154. Public Internet 1154 can be communicatively coupled to the NAT gateway 1138 contained in the control plane VCN 1116 and contained in the data plane VCN 1118. The service gateway 1136 contained in the control plane VCN 1116 and contained in the data plane VCN 1118 can be communicatively coupled to cloud services 1156.

In some embodiments, the data plane VCN 1118 can be integrated with customer tenancies 1170. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1146. Code to run the function may be executed in the VMs 1166(1)-(N), and the code may not be configured to run anywhere else on the data plane VCN 1118. Each VM 1166(1)-(N) may be connected to one customer tenancy 1170. Respective containers 1171(1)-(N) contained in the VMs 1166(1)-(N) may be configured to run the code. In this case, there can be a dual isolation (e.g., the containers 1171(1)-(N) running code, where the containers 1171(1)-(N) may be contained in at least the VM 1166(1)-(N) that are contained in the untrusted app subnet(s) 1162), which may help prevent incorrect or otherwise undesirable code from damaging the network of the IaaS provider or from damaging a network of a different customer. The containers 1171(1)-(N) may be communicatively coupled to the customer tenancy 1170 and may be configured to transmit or receive data from the customer tenancy 1170. The containers 1171(1)-(N) may not be configured to transmit or receive data from any other entity in the data plane VCN 1118. Upon completion of running the code, the IaaS provider may kill or otherwise dispose of the containers 1171(1)-(N).

In some embodiments, the trusted app subnet(s) 1160 may run code that may be owned or operated by the IaaS provider. In this embodiment, the trusted app subnet(s) 1160 may be communicatively coupled to the DB subnet(s) 1130 and be configured to execute CRUD operations in the DB subnet(s) 1130. The untrusted app subnet(s) 1162 may be communicatively coupled to the DB subnet(s) 1130, but in this embodiment, the untrusted app subnet(s) may be configured to execute read operations in the DB subnet(s) 1130. The containers 1171(1)-(N) that can be contained in the VM 1166(1)-(N) of each customer and that may run code from the customer may not be communicatively coupled with the DB subnet(s) 1130.

In other embodiments, the control plane VCN 1116 and the data plane VCN 1118 may not be directly communicatively coupled. In this embodiment, there may be no direct communication between the control plane VCN 1116 and the data plane VCN 1118. However, communication can occur indirectly through at least one method. An LPG 1110 may be established by the IaaS provider that can facilitate communication between the control plane VCN 1116 and the data plane VCN 1118. In another example, the control plane VCN 1116 or the data plane VCN 1118 can make a call to cloud services 1156 via the service gateway 1136. For example, a call to cloud services 1156 from the control plane VCN 1116 can include a request for a service that can communicate with the data plane VCN 1118.

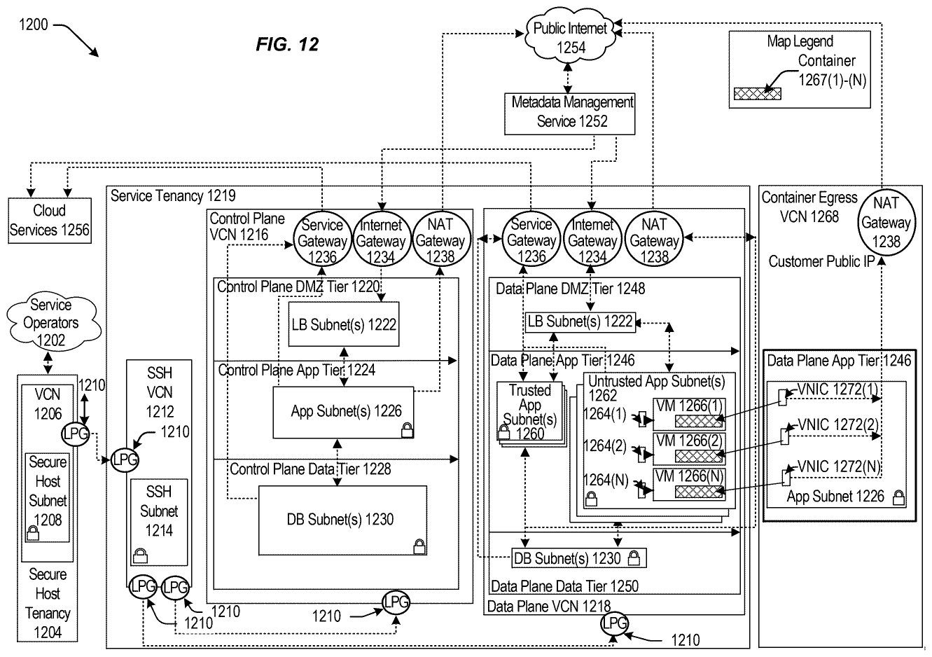

FIG. 12 is a block diagram 1200 illustrating another example pattern of an IaaS architecture, according to at least one embodiment. Service operators 1202 (e.g., service operators 902 of FIG. 9) can be communicatively coupled to a secure host tenancy 1204 (e.g., the secure host tenancy 904 of FIG. 9) that can include a virtual cloud network (VCN) 1206 (e.g., the VCN 906 of FIG. 9) and a secure host subnet 1208 (e.g., the secure host subnet 908 of FIG. 9). The VCN 1206 can include an LPG 1210 (e.g., the LPG 910 of FIG. 9) that can be communicatively coupled to an SSH VCN 1212 (e.g., the SSH VCN 912 of FIG. 9) via an LPG 1210 contained in the SSH VCN 1212. The SSH VCN 1212 can include an SSH subnet 1214 (e.g., the SSH subnet 914 of FIG. 9), and the SSH VCN 1212 can be communicatively coupled to a control plane VCN 1216 (e.g., the control plane VCN 916 of FIG. 9) via an LPG 1210 contained in the control plane VCN 1216 and to a data plane VCN 1218 (e.g., the data plane 918 of FIG. 9) via an LPG 1210 contained in the data plane VCN 1218. The control plane VCN 1216 and the data plane VCN 1218 can be contained in a service tenancy 1219 (e.g., the service tenancy 919 of FIG. 9).

The control plane VCN 1216 can include a control plane DMZ tier 1220 (e.g., the control plane DMZ tier 920 of FIG. 9) that can include LB subnet(s) 1222 (e.g., LB subnet(s) 922 of FIG. 9), a control plane app tier 1224 (e.g., the control plane app tier 924 of FIG. 9) that can include app subnet(s) 1226 (e.g., app subnet(s) 926 of FIG. 9), a control plane data tier 1228 (e.g., the control plane data tier 928 of FIG. 9) that can include DB subnet(s) 1230 (e.g., DB subnet(s) 1130 of FIG. 11). The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 934 of FIG. 9) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 9) and a network address translation (NAT) gateway 1238 (e.g., the NAT gateway 938 of FIG. 9). The control plane VCN 1216 can include the service gateway 1236 and the NAT gateway 1238.

The data plane VCN 1218 can include a data plane app tier 1246 (e.g., the data plane app tier 946 of FIG. 9), a data plane DMZ tier 1248 (e.g., the data plane DMZ tier 948 of FIG. 9), and a data plane data tier 1250 (e.g., the data plane data tier 950 of FIG. 9). The data plane DMZ tier 1248 can include LB subnet(s) 1222 that can be communicatively coupled to trusted app subnet(s) 1260 (e.g., trusted app subnet(s) 1160 of FIG. 11) and untrusted app subnet(s) 1262 (e.g., untrusted app subnet(s) 1162 of FIG. 11) of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include primary VNICs 1264(1)-(N) that can be communicatively coupled to tenant virtual machines (VMs) 1266(1)-(N) residing within the untrusted app subnet(s) 1262. Each tenant VM 1266(1)-(N) can run code in a respective container 1267(1)-(N), and be communicatively coupled to an app subnet 1226 that can be contained in a data plane app tier 1246 that can be contained in a container egress VCN 1268. Respective secondary VNICs 1272(1)-(N) can facilitate communication between the untrusted app subnet(s) 1262 contained in the data plane VCN 1218 and the app subnet contained in the container egress VCN 1268. The container egress VCN can include a NAT gateway 1238 that can be communicatively coupled to public Internet 1254 (e.g., public Internet 954 of FIG. 9).

The Internet gateway 1234 contained in the control plane VCN 1216 and contained in the data plane VCN 1218 can be communicatively coupled to a metadata management service 1252 (e.g., the metadata management system 952 of FIG. 9) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some examples, the pattern illustrated by the architecture of block diagram 1200 of FIG. 12 may be considered an exception to the pattern illustrated by the architecture of block diagram 1100 of FIG. 11 and may be desirable for a customer of the IaaS provider if the IaaS provider cannot directly communicate with the customer (e.g., a disconnected region). The respective containers 1267(1)-(N) that are contained in the VMs 1266(1)-(N) for each customer can be accessed in real-time by the customer. The containers 1267(1)-(N) may be configured to make calls to respective secondary VNICs 1272(1)-(N) contained in app subnet(s) 1226 of the data plane app tier 1246 that can be contained in the container egress VCN 1268. The secondary VNICs 1272(1)-(N) can transmit the calls to the NAT gateway 1238 that may transmit the calls to public Internet 1254. In this example, the containers 1267(1)-(N) that can be accessed in real-time by the customer can be isolated from the control plane VCN 1216 and can be isolated from other entities contained in the data plane VCN 1218. The containers 1267(1)-(N) may also be isolated from resources from other customers.

In other examples, the customer can use the containers 1267(1)-(N) to call cloud services 1256. In this example, the customer may run code in the containers 1267(1)-(N) that requests a service from cloud services 1256. The containers 1267(1)-(N) can transmit this request to the secondary VNICs 1272(1)-(N) that can transmit the request to the NAT gateway that can transmit the request to public Internet 1254. Public Internet 1254 can transmit the request to LB subnet(s) 1222 contained in the control plane VCN 1216 via the Internet gateway 1234. In response to determining the request is valid, the LB subnet(s) can transmit the request to app subnet(s) 1226 that can transmit the request to cloud services 1256 via the service gateway 1236.

It should be appreciated that IaaS architectures 900, 1000, 1100, 1200 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

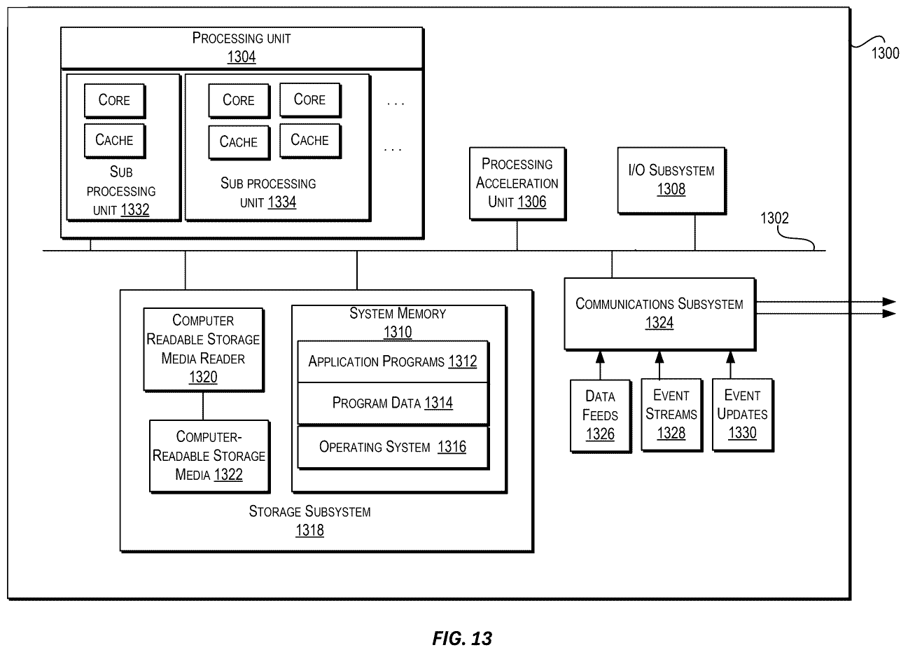

FIG. 13 illustrates an example computer system 1300, in which various embodiments may be implemented. The system 1300 may be used to implement any of the computer systems described above. As shown in the figure, computer system 1300 includes a processing unit 1304 that communicates with a number of peripheral subsystems via a bus subsystem 1302. These peripheral subsystems may include a processing acceleration unit 1306, an I/O subsystem 1308, a storage subsystem 1318 and a communications subsystem 1324. Storage subsystem 1318 includes tangible computer-readable storage media 1322 and a system memory 1310.

Bus subsystem 1302 provides a mechanism for letting the various components and subsystems of computer system 1300 communicate with each other as intended. Although bus subsystem 1302 is shown schematically as a single bus, alternative embodiments of the bus subsystem may utilize multiple buses. Bus subsystem 1302 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1304, which can be implemented as one or more integrated circuits (e.g., a conventional microprocessor or microcontroller), controls the operation of computer system 1300. One or more processors may be included in processing unit 1304. These processors may include single core or multicore processors. In certain embodiments, processing unit 1304 may be implemented as one or more independent processing units 1332 and/or 1334 with single or multicore processors included in each processing unit. In other embodiments, processing unit 1304 may also be implemented as a quad-core processing unit formed by integrating two dual-core processors into a single chip.

In various embodiments, processing unit 1304 can execute a variety of programs in response to program code and can maintain multiple concurrently executing programs or processes. At any given time, some or all of the program code to be executed can be resident in processor(s) 1304 and/or in storage subsystem 1318. Through suitable programming, processor(s) 1304 can provide various functionalities described above. Computer system 1300 may additionally include a processing acceleration unit 1306, which can include a digital signal processor (DSP), a special-purpose processor, and/or the like.

I/O subsystem 1308 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 360 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 3D scanners, 3D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1300 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1300 may comprise a storage subsystem 1318 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1304 provide the functionality described above. Storage subsystem 1318 may also provide a repository for storing data used in accordance with the present disclosure.

As depicted in the example in FIG. 13, storage subsystem 1318 can include various components including a system memory 1310, computer-readable storage media 1322, and a computer readable storage media reader 1320. System memory 1310 may store program instructions that are loadable and executable by processing unit 1304. System memory 1310 may also store data that is used during the execution of the instructions and/or data that is generated during the execution of the program instructions. Various different kinds of programs may be loaded into system memory 1310 including but not limited to client applications, Web browsers, mid-tier applications, relational database management systems (RDBMS), virtual machines, containers, etc.

System memory 1310 may also store an operating system 1316. Examples of operating system 1316 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1300 executes one or more virtual machines, the virtual machines along with their guest operating systems (GOSs) may be loaded into system memory 1310 and executed by one or more processors or cores of processing unit 1304.

System memory 1310 can come in different configurations depending upon the type of computer system 1300. For example, system memory 1310 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1310 may include a basic input/output system (BIOS) containing basic routines that help to transfer information between elements within computer system 1300, such as during start-up.

Computer-readable storage media 1322 may represent remote, local, fixed, and/or removable storage devices plus storage media for temporarily and/or more permanently containing, storing, computer-readable information for use by computer system 1300 including instructions executable by processing unit 1304 of computer system 1300.

Computer-readable storage media 1322 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1322 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1322 may include, but is not limited to, Zip® drives, flash memory cards, universal serial bus (USB) flash drives, secure digital (SD) cards, DVD disks, digital video tape, and the like. Computer-readable storage media 1322 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1300.

Machine-readable instructions executable by one or more processors or cores of processing unit 1304 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1324 provides an interface to other computer systems and networks. Communications subsystem 1324 serves as an interface for receiving data from and transmitting data to other systems from computer system 1300. For example, communications subsystem 1324 may enable computer system 1300 to connect to one or more devices via the Internet. In some embodiments communications subsystem 1324 can include radio frequency (RF) transceiver components for accessing wireless voice and/or data networks (e.g., using cellular telephone technology, advanced data network technology, such as 3G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1324 can provide wired network connectivity (e.g., Ethernet) in addition to or instead of a wireless interface.

In some embodiments, communications subsystem 1324 may also receive input communication in the form of structured and/or unstructured data feeds 1326, event streams 1328, event updates 1330, and the like on behalf of one or more users who may use computer system 1300.

By way of example, communications subsystem 1324 may be configured to receive data feeds 1326 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1324 may also be configured to receive data in the form of continuous data streams, which may include event streams 1328 of real-time events and/or event updates 1330, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1324 may also be configured to output the structured and/or unstructured data feeds 1326, event streams 1328, event updates 1330, and the like to one or more databases that may be in communication with one or more streaming data source computers coupled to computer system 1300.

Computer system 1300 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1300 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving, by a computer, a query to provide a summary of patient-specific information regarding a condition for a particular patient;
determining, by the computer, a category for the query;
retrieving, by the computer, data relevant to the query from an electronic health record (EHR) database, wherein the data as retrieved includes at least structured and unstructured content;
processing, by the computer, the data as retrieved based on the category;
filtering, by the computer, the data as processed based on the category;
generating, by a generative machine learning model on the computer, a narrative summary including a first portion of the data as filtered including at least a portion of the unstructured content;
generating, by the computer, a structured summary including a second portion of the data as filtered including at least a portion of the structured content; and
formatting, by the computer, the narrative summary and the structured summary into an output, wherein
determining the category for the query includes selecting the category from a plurality of categories including at least one of New Admit, New to Me, and Rounded on Before, and
processing performed for a first selected category differs from processing performed for a second selected category.

2. The computer-implemented method of claim 1, wherein processing includes,
for the first selected category, providing a first set of processing modules, and
for the second selected category, providing a second set of processing modules.

3. The computer-implemented method of claim 1, wherein filtering includes considering the data as processed according to a semantic knowledge graph selected based on the category.

4. The computer-implemented method of claim 3, wherein the data is processed according to the semantic knowledge graph by applying enrichment that prioritizes selected data in a predefined hierarchy model based on the category and at least one of a reason for visit and a chief complaint for the particular patient.

5. The computer-implemented method of claim 4, wherein the predefined hierarchy model includes prioritizing data related to changes in the condition for the particular patient during a predetermined time window.

6. The computer-implemented method of claim 1, further comprising:
transforming the data into an intermediate representation normalized to clinical terminologies, and
filtering the intermediate representation to meet a token budget for the generative machine learning model on the computer.

7. The computer-implemented method of claim 6, further comprising:
caching the intermediate representation as keyed to at least a selected one of the particular patient, the category, and a time window, and
reusing the cache in responding to updated queries related to the particular patient and the category.

8. The computer-implemented method of claim 1, wherein processing includes,
for the unstructured content, processing the unstructured content through at least one of optical character recognition, image recognition, and chunking processes.

9. The computer-implemented method of claim 1, further comprising
determining a role of an originator of the query, the role determining a level of permissions assigned to the originator,
wherein processing is modified according to the role as determined.

10. A system comprising:
a computer comprising one or more processors and one or more computer-readable media storing instructions which, when executed by the one or more processors, cause the computer to at least:
receive a query to provide a summary of patient-specific information regarding a condition for a particular patient;
determine a category for the query, wherein determining includes selecting the category from a plurality of categories including at least one of New Admit, New to Me, and Rounded on Before;
retrieve data relevant to the query from an electronic health record (EHR) database, wherein the data as retrieved includes at least structured and unstructured content;
process the data as retrieved based on the category;
filter the data as processed based on the category;
generate, by a generative machine learning model on the computer, a narrative summary including a first portion of the data as filtered including at least a portion of the unstructured content;
generate a structured summary including a second portion of the data as filtered including at least a portion of the structured content; and
format the narrative summary and the structured summary into an output,
wherein processing performed for a first selected category differs from processing performed for a second selected category.

11. The system of claim 10, wherein processing includes,
for the first selected category, providing a first set of processing modules, and
for the second selected category, providing a second set of processing modules.

12. The system of claim 10, wherein filtering includes considering the data as processed according to a semantic knowledge graph and a hierarchy model selected based on the category by applying enrichment that prioritizes selected data based on the category and at least one of a reason for visit and a chief complaint for the particular patient.

13. The system of claim 12, wherein the hierarchy model includes prioritization of data related to changes in the condition for the particular patient during a predetermined time window.

14. The system of claim 10, the instructions further causing the computer to
transform the data into an intermediate representation normalized to clinical terminologies, and
filter the intermediate representation to meet a token budget for the generative machine learning model.

15. The system of claim 14, the instructions further causing the computer to
cache the intermediate representation as keyed to at least a selected one of the particular patient, the category, and a time window, and
reuse the cache in responding to updated queries related to the particular patient and the category.

16. The system of claim 10, wherein processing includes,
for the unstructured content, processing the unstructured content through at least one of optical character recognition, image recognition, and chunking processes.

17. One or more non-transitory computer-readable media storing instructions which, when executed by one or more processors on a computer, cause the computer to at least:
receive a query to provide a summary of patient-specific information regarding a condition for a particular patient;
determine a category for the query, wherein determining includes selecting the category from a plurality of categories including at least one of New Admit, New to Me, and Rounded on Before;
retrieve data relevant to the query from an electronic health record (EHR) database, wherein the data as retrieved includes at least structured and unstructured content;
process the data as retrieved based on the category;
filter the data as processed based on the category;
generate, by a generative machine learning model on the computer, a narrative summary including a first portion of the data as filtered including at least a portion of the unstructured content;
generate a structured summary including a second portion of the data as filtered including at least a portion of the structured content; and
format the narrative summary and the structured summary into an output,
wherein processing performed for a first selected category differs from processing performed for a second selected category.

18. The one or more non-transitory computer-readable media of claim 17, wherein processing includes,
for the first selected category, providing a first set of processing modules, and
for the second selected category, providing a second set of processing modules.

19. The one or more non-transitory computer-readable media of claim 17, wherein filtering includes considering the data as processed according to a semantic knowledge graph and a hierarchy model selected based on the category by applying enrichment that prioritizes selected data based on the category and at least one of a reason for visit and a chief complaint for the particular patient.

20. The one or more non-transitory computer-readable media of claim 17, the instructions further causing the computer to
transform the data into an intermediate representation normalized to clinical terminologies, and
filter the intermediate representation to meet a token budget for the generative machine learning model.
